(19) United States
(12) Patent Application Publication
(10) Pub. No.: US 2026/0166097 A1
(43) Pub. Date: Jun. 18, 2026

(54) METHODS AND COMPOSITIONS CONCERNING BACTEROIDES OVATUS

(71) Applicant: BOARD OF REGENTS, THE UNIVERSITY OF TEXAS SYSTEM, Austin, TX (US)

(72) Inventors: Robert R. JENQ, Houston, TX (US); Eiko HAYASE, Houston, TX (US)

(73) Assignee: BOARD OF REGENTS, THE UNIVERSITY OF TEXAS SYSTEM, Austin, TX (US)

(21) Appl. No.: 19/129,290

(22) PCT Filed: Nov. 10, 2023
(86) PCT No.: PCT/US2023/079441

§ 371 Date: May 12, 2025

Related U.S. Application Data
(60) Provisional application No. 63/424,801, filed on Nov. 11, 2022.

(51) Int. Cl. A61K 35/741; A61K 31/4164; A61K 31/42; A61K 31/43; A61K 31/505; A61K 31/5377; A61K 31/546; A61K 31/7056; A61K 31/717; A61K 35/00; A61K 38/14; A61K 45/06; A61P 37/06

(52) U.S. Cl. CPC A61K 35/741; A61K 31/4164; A61K 31/42; A61K 31/43; A61K 31/505; A61K 31/5377; A61K 31/546; A61K 31/7056; A61K 31/717; A61K 38/14; A61K 45/06; A61P 37/06; A61K 2035/115

(57) ABSTRACT

Compositions and methods involving Bacteroides ovatus are provided herein in certain aspects to reduced GVHD, including GVHD associated with Bacteroides thetaiotaomicron. This includes therapeutic compositions comprising the bacteria Bacteroides ovatus as well as methods of administering Bacteroides ovatus to a patient in need thereof.

This application claims priority to and the benefit of U.S. Provisional Application No. 63/424,801 filed Nov. 11, 2022, the contents of which are incorporated into the present application by reference in its entirety.

This invention was made with government support under HL124112 and CA016672 awarded by the National Institutes of Health. The government has certain rights in the invention.

The application contains a Sequence Listing in compliance with ST.26 format and is hereby incorporated by reference in its entirety. Said Sequence Listing, created on Nov. 10, 2023, is named MDACP1348WO_xml and is 5,403 bytes in size.

### BACKGROUND

### I. Field of the Disclosure

This disclosure relates to the field of microbiology, oncology, and medicine.

### II. Background

Graft-versus-host disease (GVHD) is a common complication in patients undergoing allogeneic hematopoietic stem cell transplantation (allo-HSCT) and occurs when donor T cells recognize a patient's tissues as foreign. The intestine is often targeted, and severe acute lower gastrointestinal GVHD (aGI-GVHD) tends to have a poor prognosis because of poor treatment response to GVHD¹. Approximately half of aGI-GVHD cases do not respond to first-line steroid therapy, leading to a high risk for severe complications and reduced overall survival^{2,3}. Novel immune suppression strategies to treat steroid-refractory GVHD have been established, including Janus kinase 1/2 (JAK1/2) inhibitors, with demonstrated clinical efficacy, though not all patients will respond^{4,5}.

The intestinal microbiota is an important modulator of the host immune system^{6,7} and modulates the pathophysiology of GVHD⁸. Patients undergoing allo-HSCT are at high risk for perturbations in the intestinal microbiota resulting from a number of factors, chief amongst them exposure to antibiotics for prevention and treatment of bacterial infections post-transplant. Broad-spectrum antibiotics such as carbapenems have been reported to increase the incidence of aGI-GVHD⁹⁻¹². Recently, fecal microbiota transplantation has been shown to result in improvement in GVHD in steroid-refractory patients¹³⁻¹⁵, suggesting that the intestinal microbiota can modulate aGI-GVHD treatment responsiveness. It remains unclear, however, how intestinal microbial composition can modulate treatment response of aGI-GVHD.

In allo-HSCT, antibiotics for prophylaxis and treatment of bacterial infections are essential owing to the high risks of these infections, but they can disrupt the intestinal microbiome. Broad-spectrum antibiotics such as carbapenems have been reported to increase the incidence of aGI-GVHD (Farowski et al., 2018; Hidaka et al., 2018; Lee et al., 2019; Shono et al., 2016). Thus, suitable treatments or prophylaxis of intestinal disorders from HSCT and/or antibiotic treatments are needed.

### SUMMARY OF THE INVENTION

Compositions and methods involving Bacteroides ovatus are provided herein. This includes therapeutic compositions comprising the bacteria Bacteroides ovatus as well as methods of administering Bacteroides ovatus to a patient in need thereof.

The therapeutic composition may comprise Bacteroides ovatus in a unit dosage. The unit dosage may be between 1×10⁵ to 9×10⁸ colony forming units (CFU) of the Bacteroides ovatus. In some aspects, the therapeutic composition comprises a polysaccharide. In certain aspects, the therapeutic composition does not comprise a polysaccharide. The polysaccharide may or may not comprise a dietary-derived polysaccharide. The polysaccharide may or may not comprise a xylose-comprising polysaccharide. The xylose-comprising polysaccharide may or may not comprise arabinoxylan, which may or may not be a cereal grain arabinoxylan. The arabinoxylan may or may not comprise wheat arabinoxylan. The xylose-comprising polysaccharide may or may not comprise xyloglucan, which may or may not be a XXGG-type and/or a XXXG-type xyloglucan. The xyloglucan may or may not comprise tamarind xyloglucan.

Certain aspects concern methods of treating a disease a patient. In some aspects, the method comprises the step of administering to the patient a therapeutically effective amount of any therapeutic composition described herein. In some aspects, the patient is administered a composition comprising Bacteroides ovatus and a polysaccharide, including any polysaccharide described herein. The polysaccharide may or may not be administered concurrently with the Bacteroides ovatus. The polysaccharide may or may not be administered sequentially with the Bacteroides ovatus. The polysaccharide may or may not comprise a dietary-derived polysaccharide. The polysaccharide may or may not comprise a xylose-comprising polysaccharide. The xylose-comprising polysaccharide may or may not comprise arabinoxylan, which may or may not comprise wheat arabinoxylan. The xylose-comprising polysaccharide may or may not comprise xyloglucan, which may or may not comprise tamarind xyloglucan.

In certain aspects, the disease is an intestinal disease. In some aspects, the intestinal disease comprises acute gastrointestinal graft versus host disease, an antibiotic-mediated microbiome injury, or intestinal inflammation. In certain aspects, the patient is determined to be steroid refractory. In other aspects the patient is determined to react positively to treatment with one or more steroids. A steroid treatment is with prednisone or methylprednisone but is not limited to prednisone or methylprednisone, as other steroids may be used. The antibiotic-mediated microbiome injury may occur when the patient has or will receive an antibiotic. The antibiotic may comprise one or more antibiotic compounds, including a broad spectrum antibiotic. In some aspects, the antibiotic is a broad spectrum antibiotic. In some aspects, the antibiotic comprises cefepime, daptomycin, linezolide, penicillin, metronidazole, sulfamethoxazole, trimethoprim, vancomycin, and/or clindamycin. In some aspects, the antibiotic does not comprise cefepime, daptomycin, linezolide, penicillin, metronidazole, sulfamethoxazole, trimethoprim, vancomycin, and/or clindamycin. In certain aspects, the antibiotic comprises a carbapenem, a macrolide, a quinolone, and/or an aminoglycoside. In some aspects, the carbapenem comprises meropenem. In certain aspects, the antibiotic does not comprise a carbapenem, a macrolide, a quinolone, and/or an aminoglycoside.

Certain aspects concern methods of preventing complications from hematopoietic stem cell transplantation in a patient. In some aspects, the method comprises the step of administering to the patient a therapeutically effective amount of any therapeutic composition described herein. In some aspects, the patient is administered a composition comprising Bacteroides ovatus and a polysaccharide, including any polysaccharide described herein. The polysaccharide may or may not be administered concurrently with the Bacteroides ovatus. The polysaccharide may or may not be administered sequentially with the Bacteroides ovatus. The polysaccharide may or may not comprise a dietary-derived polysaccharide. The polysaccharide may or may not comprise a xylose-comprising polysaccharide. The xylose-comprising polysaccharide may or may not comprise arabinoxylan, which may or may not comprise wheat arabinoxylan. The xylose-comprising polysaccharide may or may not comprise xyloglucan, which may or may not comprise tamarind xyloglucan.

In certain aspects, the hematopoietic stem cell transplantation comprises allogeneic hematopoietic stem cell transplantation.

Certain aspects concern methods for inducing mucus degradation in intestines of a patient and/or reducing Bacteroides thetaiotaomicron (BT) in a patient. In some aspects, the method comprises the step of administering to the patient a therapeutically effective amount of any therapeutic composition described herein. In some aspects, the patient is administered a composition comprising Bacteroides ovatus and a polysaccharide, including any polysaccharide described herein. The polysaccharide may or may not be administered concurrently with the Bacteroides ovatus. The polysaccharide may or may not be administered sequentially with the Bacteroides ovatus. The polysaccharide may or may not comprise a dietary-derived polysaccharide. The polysaccharide may or may not comprise a xylose-comprising polysaccharide. The xylose-comprising polysaccharide may or may not comprise arabinoxylan, which may or may not comprise wheat arabinoxylan. The xylose-comprising polysaccharide may or may not comprise xyloglucan, which may or may not comprise tamarind xyloglucan.

Certain aspects concern methods comprising administering to a patient any of the therapeutic composition described herein. In some aspects, the patient is at risk of developing graft versus host disease (GVHD), which may be acute gastrointestinal GVHD. A patient is at risk for developing GVHD if they will be receiving biological material that may trigger an immune response. In some aspects, the patient has, or is at risk of developing, an intestinal disease. In certain aspects, the intestinal disease is an antibiotic-mediated microbiome injury or intestinal inflammation. In some aspects, when the disease is an antibiotic-mediated microbiome injury, the patient has received or will receive an antibiotic. The antibiotic may one or more antibiotic compounds, including a broad spectrum antibiotic. In some aspects, the antibiotic is a broad spectrum antibiotic. In some aspects, the antibiotic comprises cefepime, daptomycin, linezolide, penicillin, metronidazole, sulfamethoxazole, trimethoprim, vancomycin, and/or clindamycin. In some aspects, the antibiotic does not comprise cefepime, daptomycin, linezolide, penicillin, metronidazole, sulfamethoxazole, trimethoprim, vancomycin, and/or clindamycin. In certain aspects, the antibiotic comprises a carbapenem, a macrolide, a quinolone, and/or an aminoglycoside. In some aspects, the carbapenem comprises meropenem. In certain aspects, the antibiotic does not comprise a carbapenem, a macrolide, a quinolone, and/or an aminoglycoside. In some aspects, the patient has received or will receive an antibiotic. The antibiotic may one or more antibiotic compounds, including a broad spectrum antibiotic. In some aspects, the antibiotic is a broad spectrum antibiotic. In some aspects, the antibiotic comprises cefepime, daptomycin, linezolide, penicillin, metronidazole, sulfamethoxazole, trimethoprim, vancomycin, and/or clindamycin. In some aspects, the antibiotic does not comprise cefepime, daptomycin, linezolide, penicillin, metronidazole, sulfamethoxazole, trimethoprim, vancomycin, and/or clindamycin. In certain aspects, the antibiotic comprises a carbapenem, a macrolide, a quinolone, and/or an aminoglycoside. In some aspects, the carbapenem comprises meropenem. In certain aspects, the antibiotic does not comprise a carbapenem, a macrolide, a quinolone, and/or an aminoglycoside. In some aspects, the patient has received or will receive a stem cell treatment. The stem cell treatment may or may not comprise a hematopoietic stem cell transplantation, which may or may not comprise a allogeneic hematopoietic stem cell transplantation.

In some aspects, the patient administered a therapeutic composition has an altered microbiome profile compared to a standard. In some aspects, the microbiome profile comprises a less diverse microbiome profile than a standard. In certain aspects, the altered microbiome profile comprises a higher abundance of Enterococcus, Citrobacter, Streptococcus, Staphylococcus, and/or Enterobacter bacteria compared to a standard. In some aspects, the altered microbiome profile comprises a lower abundance of Bacteroides, Prevotella, Faecalibacterium, and/or UBA1819 bacteria compared to a standard. In certain aspects, the standard comprises a microbiome profile of a healthy individual. The standard may also comprise a microbiome profile of a patient found responsive to a steroid.

Certain aspects relate to methods for prognosing responsiveness to a steroid therapy in a patient, the method comprising the steps of; obtaining a microbiome profile from the patient; and comparing the microbiome profile to a standard, wherein the patient has, or will receive, a stem cell therapy. In some aspects, the standard comprises a microbiome profile of a healthy individual. In certain aspects, the standard comprises a microbiome profile of a patient found responsive to a steroid. In certain aspects, the patient is prognosed to be steroid-refractory when the microbiome profile from the patient comprises a higher abundance of Enterococcus, Citrobacter, Streptococcus, Staphylococcus, and/or Enterobacter bacteria compared to the standard. In some aspects, the patient is prognosed to be steroid-refractory when the microbiome profile from the patient comprises a lower abundance of Bacteroides, Prevotella, Faecalibacterium, and/or UBA1819 bacteria compared to the standard. In some aspects, the patient is prognosed to be steroid-refractory when the microbiome profile is less diverse compared to the standard.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description, Claims, and description of drawing.

Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific aspects presented herein.

FIGS. 1A-1G show the high abundance of Bacteroides was associated with steroid-responsive GVHD. (a) Cluster dendrogram analyzed using H-clustering of weighted UniFrac. (b) The microbiome composition shown as stacked bar graphs. (c) PCoA of fecal samples collected from healthy volunteers or each cluster of aGI-GVHD patients (“Healthy” cluster—upper left, “Cluster 1”—lower left, “Cluster 2”—upper right). (d) Distances from healthy volunteers in weighted UniFrac. (e) Numbers of patients with steroid-responsive and -refractory GVHD. (f) Proportions of patients with steroid-responsive and -refractory GVHD. (g) Volcano plot of differentially abundant genera between clusters 1 and 2.

FIGS. 2A-2F show steroid-refractory aGI-GVHD patients showed significantly dysbiotic intestinal microbiome than steroid-responsive aGI-GVHD patients. (a-f) The intestinal microbiome analyzed by 16S rRNA sequencing in patient stool samples collected at presentation with aGI-GVHD. (a) Alpha diversity shown as Shannon index. (b) Principal coordinates analysis (PCoA) of fecal samples collected from healthy volunteers (left) or steroid-responsive (middle) or steroid-refractory (right) patients. (c) Distances from healthy volunteers in weighted UniFrac. (d) The composition of the intestinal microbiome. (e) Volcano plot of differentially abundant genera. (f) Relative abundance of genera that were significantly different between steroid-responsive (left) and -refractory (right) aGI-GVHD.

FIGS. 3A-3G show the high abundance of Bacteroides ovatus and B. ovatus-derived pathways were associated with steroid-responsive GVHD. (a) Graphical summary of antibiotics used in individual patients. (b) Proportions of patients with antibiotic exposure between hematopoietic stem cell transplant (HSCT) and the onset of GVHD (responsive on left side of bar, refractory on right side of bar). (c) Univariate logistic regression analysis for the risk of antibiotic exposure on steroid refractory GVHD. (d) Proportions of patients with carbapenem exposure with or without daptomycin (left) and those with daptomycin exposure with or without carbapenem (right). (e-g) Data analyzed by shotgun sequencing of fecal samples collected from aGI-GVHD patients (steroid-responsive; n=11, steroid-refractory; n=12). (e) Volcano plot of differentially abundant species between steroid-responsive and -refractory GVHD. (f) Volcano plot of differentially abundant pathways of the genus Bacteroides. (g) The top 50 subclasses of differentially abundant pathways of the genus Bacteroides.

FIGS. 4A-4M show Bacteroides ovatus improved GVHD-related mortality in meropenem-aggravated colonic GVHD. (a) Experimental schema of murine GVHD model using meropenem treatment followed by oral gavage of 20 million colony-forming units of B. ovatus daily for 3 days. (b) Overall survival after allo-HSCT. (Allogeneic+meropenem: top line, Allogeneic+meropenem+B. ovatus: bottom line) Data are combined from two independent experiments. (c) Bacterial densities of mouse stool samples collected on day 21. Bacterial densities were measured by 16S rRNA gene qPCR. (d) Alpha diversity, measured by the Shannon index, was quantified in fecal samples. (e) Principal coordinates analysis (PCoA) of fecal samples (Allogeneic+meropenem: left, Allogeneic+meropenem+B. ovatus: right). (f) Bacterial genera composition of fecal samples. (g) Volcano plot of differentially abundances of zotu. (h) Relative abundance of B. ovatus (left), B. theta (middle) and A. muciniphila (right). (i) Absolute abundance of B. ovatus (left), B. theta (middle) and A. muciniphila (right). (j) Relative abundance of B. ovatus in mouse stool samples collected on days 21 and 28. (k) Periodic acid-Schiff (PAS) staining of histological colon sections collected on day 23. Bar, 100 m. The areas inside dotted lines indicate the inner dense colonic mucus layer. (1) Mucus thickness on day 23. Data are shown from one representative experiment. (m) GVHD histology scores of the colon harvested on day 28. GVHD histology scores were quantified by a blinded pathologist.

FIGS. 5A-5C show mucolytic activity of Bacteroides thetaiotaomicron and Akkermansia muciniphila are suppressed in meropenem-treated mice by administration of Bacteroides ovatus. (a) Heatmap showing scaled relative expression levels of polysaccharide utilization loci (PULs) in B. theta RNA transcripts sequenced from stool collected from meropenem-treated allo-HSCT mice with or without administration of B. ovatus on day 21. Right: Significantly altered PULs and their substrates. (b) Heatmap showing scaled relative expression levels of carbohydrate-active enzymes (cazymes) in A. muciniphila RNA transcripts sequenced from stool collected from meropenem-treated allo-HSCT mice with or without administration of B. ovatus on day 21. (c) Relative abundances of monosaccharides of supernatants from colonic luminal content collected from meropenem-treated allo-HSCT mice with or without administration of B. ovatus on day 23 (Allogeneic+meropenem: left bar, Allogeneic+meropenem+B. ovatus: right bar) measured by ion chromatography-mass spectrometry (IC-MS). Combined data from two independent experiments are shown as means±SEM.

FIGS. 6A-6I show degradation of xylose-comprising polysaccharides by Bacteroides ovatus suppressed mucus-degrading functionality by Bacteroides thetaiotaomicron. (a) Experimental schema of in vitro bacterial culture assay using B. ovatus (MDA-HVS B0001) cultured in minimum nutrition medium with each polysaccharide and B. theta (MDA-JAX BT001) cultured in BYEM10 with porcine gastric mucin. (b) Concentrations of porcine gastric mucin in the culture supernatant were determined using a PAS-based colorimetric assay. Combined data from two independent experiments are shown as means±SEM. (c) Relative abundances of monosaccharides of the B. ovatus culture supernatant with each polysaccharide measured by ion chromatography-mass spectrometry (IC-MS). (d) Experimental schema of gnotobiotic model using introduction of 20 million colony-forming units of B. ovatus (MDA-HVS B0001). (e) Heatmap showing scaled relative expression levels of polysaccharide utilization loci (PULs) in B. theta RNA transcripts sequenced from stool collected from B. theta (ATCC 29148)-colonized gnotobiotic mice with or without co-administration of B. ovatus. Transcripts were evaluated on day 14 after bacterial introduction to germ-free mice. Right: Significantly altered PULs and their substrates. (f) Experimental schema of gnotobiotic model using introduction of 20 million colony-forming units of wild-type B. ovatus (ATCC8483 with gene deletion of thymidine kinase) or xylan-PUL deficient B. ovatus. (g) Relative abundances of xylose of supernatants from colonic luminal content collected from gnotobiotic mice with administration of wild-type B. ovatus (ATCC8483 with gene deletion of thymidine kinase) or xylan-PUL deficient B. ovatus on day 14 measured by ion chromatography-mass spectrometry (IC-MS). (h) Experimental schema of murine GVHD model using meropenem treatment followed by oral gavage of 20 million colony-forming units of of wild-type B. ovatus (ATCC8483 with gene deletion of thymidine kinase) or xylan-PUL deficient B. ovatus daily for 3 days. (i) Overall survival after allo-HSCT (Allogeneic+meropenem: bottom line, Allogeneic+meropenem+B. ovatus wild type: top line, Allogeneic+meropenem+B. ovatus xylan PUL deficient: middle line). Data are combined from three independent experiments.

FIGS. 7A-7B show aGI-GVHD patients showed reduced abundances of the genera Prevotella and Faecalibacterium. (a) PCoA of fecal samples collected from healthy volunteers or aGI-GVHD patients. (b) Volcano plot of differentially abundant genera analyzed by 16S rRNA gene sequencing of fecal samples compared between healthy volunteers and aGI-GVHD patients.

FIGS. 8A-8D show Bacteroides ovatus was associated with steroid responsive GVHD in the validation cohort. (a) Relative abundance of the genus Bacteroides at the onset of aGI-GVHD. (b) Relative abundance of Bacteroides ovatus at the onset of aGI-GVHD. (a, b) Differentially abundant taxa were analyzed using DESeq2. (c) Venn diagram that shows species that were significantly different abundances between steroid-responsive and steroid-refractory patients in discovery cohort and validation cohort. (d) Relative abundance of Bacteroides ovatus in aGI-GVHD patient stool samples collected on day 14 after allo-HSCT.

FIGS. 9A-9F show Bacteroides ovatus did not show mucus-degrading functionality like B. theta. (a) Circular plot of open reading frames (ORFs) derived from the complete genome (MDA-HVS B0001). Blue and green bars represent ORFs on the plus strand and the minus strand, respectively. Inner purple-olive ring depicts degree of GC skewing. (b) Experimental schema of building and analyzing metagenome-assembled genomes (MAGs) from the 3 patient stool datasets. (c) Calculated mean average distances between assembly within each cluster group. (d) Distances between clusters. (e) Experimental schema of in vitro bacterial culture assay of B. theta (MDA-JAX BT001) or B. ovatus (MDA-HVS B0001) in media with porcine gastric mucin-containing medium. (f) Relative concentrations of porcine gastric mucin in medium following culture with B. theta (MDA-JAX BT001) or B. ovatus (MDA-HVS B0001). B. theta or B. ovatus was first introduced to porcine gastric mucin-containing medium. At 24 hours of culture, levels of mucin glycans in the culture supernatant were determined using a colorimetric assay.

FIGS. 10A-10K show introduction of Bacteroides ovatus did not alter abundance and functionality of B. theta in meropenem-untreated allo-HSCT mice. (a) Experimental schema of murine GVHD model with oral gavage of 20 million colony-forming units of B. ovatus daily from days 16 to 18. (b) Overall survival after allo-HSCT. Data are combined from two independent experiments. (c) Bacterial densities of mouse stool samples collected on day 21. Bacterial densities were measured by 16S rRNA gene qPCR. (d) Alpha diversity, measured by the Shannon index, was quantified in fecal samples. (e) Principal coordinates analysis (PCoA) of fecal samples. (f) Bacterial genera composition of fecal samples. (g) Volcano plot of differentially abundances of zotu. (h) Relative abundance of B. ovatus (left), B. theta (middle) and A. muciniphila (right). (i) Absolute abundance of B. ovatus (left), B. theta (middle) and A. muciniphila (right). (b-i) Combined data from two independent experiments. (j) PAS staining of histological colon sections collected on day 23. Bar, 100 m. The areas inside dotted lines indicate the inner dense colonic mucus layer. (k) Mucus thickness on day 23. Data are shown from one representative experiment.

FIGS. 11A-11D show introduction of Bacteroides ovatus increased fecal levels of soluble monosaccharides in mice monocolonized with B. ovatus. (a) Heatmap showing scaled relative expression levels of polysaccharide utilization loci (PULs) in B. theta RNA transcripts sequenced from stool collected from meropenem-untreated allo-HSCT mice with or without administration of B. ovatus on day 21. Right: PULs and their modularity and substrate names. (b) Relative abundances of monosaccharides of supernatants from colonic luminal content collected from germ-free (GF) mice with (right bar) or without (left bar) administration of B. ovatus on day 14 measured by IC-MS. Data are shown from one representative experiment. (c) Relative abundances of monosaccharides of supernatants from colonic luminal content collected from meropenem-untreated allo-HSCT mice with or without administration of B. ovatus on day 23 measured by ion chromatography-mass spectrometry (IC-MS). Data are shown from one representative experiment. (d) Absolute abundances of tryptophan metabolites of supernatants from colonic luminal content collected from meropenem-treated allo-HSCT mice with (right bar) or without (left bar) administration of B. ovatus on day 23 measured by liquid chromatography coupled with high-resolution mass spectrometry (LC-HRMS). Data are combined from two independent experiments and are shown as means±SEM.

FIGS. 12A-12E show (a) Relative expression levels of PULs in B. ovatus RNA transcripts sequenced from stool collected from allo-HSCT mice treated or untreated with meropenem on day 28. Right: PULs and their modularity and substrate names. (b) Relative abundances of monosaccharides of supernatants from colonic luminal content collected from meropenem-untreated (left bar) or -treated (right bar) allo-HSCT mice with administration of B. ovatus on day 23 measured by IC-MS. These data were reconstituted from FIGS. 5C and 11C. (c) The correlation network analysis of B. ovatus RNA transcripts and B. theta RNA transcripts sequenced from stool collected on day 21 from meropenem-treated and -untreated allogeneic mice with administration of B. ovatus. Only negatively correlated networks are shown. (d) Experimental schema of in vitro bacterial culture assay using B. ovatus (MDA-HVS B0001) cultured in minimum nutrition medium with each polysaccharide and A. muciniphila (MDA-JAX AM001) cultured in BYEM10 with porcine gastric mucin. (e) Concentrations of porcine gastric mucin in the culture supernatant were determined using a PAS-based colorimetric assay. Data are shown from one representative experiment as means±SEM.

### DETAILED DESCRIPTION

### I. Definitions

Throughout this disclosure, “Bacteroides ovatus” and “MDA-HVS B0001” may refer to a bacteria strain that is significantly related to the Bacteroides ovatus of the American Type Culture Collection (ATCC) type strain of Bacteroides ovatus (ATCC 8483). The strain may be significantly related when it has more than 97%, 98%, 99%, 99.1%, 99.2%, 99.3%, 99.4%, 99.5%, 99.6%, 99.7%, 99.8%, 99.9%, or any range derivable therein, sequence identity to ATCC 8483. The sequence identity comparison may be a 16S, 23S, and/or total genome sequence identity comparison. In certain aspects, the Bacteroides ovatus has a gene that has more than 97%, 98%, 99% sequence identity to one or more of SEQ ID NOs. 1-4.

SEQ ID NO. 1:
tacggaggatccgagcgttatccggatttattgggtttaaagggagcgta
ggtggattgttaagtcagttgtgaaagtttgcggctcaaccgtaaaattg
cagttgaaactggcagtcttgagtacagtagaggtgggcggaattcgtgg
tgtagcggtgaaatgcttagatatcacgaagaactccgattgcgaaggca
gctcactagactgttactgacactgatgctcgaaagtgtgggtatcaaac
ang
SEQ ID NO. 2:
tacggaggatccgagcgttatccggatttattgggtttaaagggagcgta
ggtggattgttaagtcagttgtgaaagtttgcggctcaaccgtaaaattg
cagttgaaactggcagtcttgagtacagtagaggtgggcggaattcgtgg
tgtagcggtgaaatgcttagatatcacgaagaactccgattgcgaaggca
gctcactagactgtcactgacactgatgctcgaaagtgtgggtatcaaac
ang
SEQ ID NO. 3:
tacggaggatccgagcgttatccggatttattgggtttaaagggagcgta
ggtggattgttaagtcagttgtgaaagtttgcggctcaaccgtaaaattg
cagttgatactggatgtcttgagtgcagttgaggcaggcggaattcgtgg
tgtagcggtgaaatgcttagatatcacgaagaactccgattgcgaaggca
gctcactagactgttactgacactgatgctcgaaagtgtgggtatcaaac
ang
SEQ ID NO. 4:
tacgtagggggcaagcgttatccggatttattgggtttaaagggagcgta
ggtggattgttaagtcagttgtgaaagtttgcggctcaaccgtaaaattg
cagttgaaactggcagtcttgagtacagtagaggtgggcggaattcgtgg
tgtagcggtgaaatgcttagatatcacgaagaactccgattgcgaaggca
gctcactagactgttactgacactgatgctcgaaagtgtgggtatcaaac
agg

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

The term “isolated” encompasses a bacterium or other entity or substance that has been (1) separated from at least some of the components with which it was associated when initially produced (whether in nature or in an experimental setting), and/or (2) produced, prepared, purified, and/or manufactured by the hand of man. Isolated bacteria may be separated from at least about 10%, about 20%, about 30%, about 40%, about 50%, about 60%, about 70%, about 80%, about 90%, or any range derivable therein, or more of the other components with which they were initially associated. In some aspects, isolated bacteria are more than about 80%, about 85%, about 90%, about 91%, about 92%, about 93%, about 94%, about 95%, about 96%, about 97%, about 98%, about 99%, or any range derivable therein, or more than about 99% pure. As used herein, a substance is “pure” if it is substantially free of other components.

The terms “purify,” “purifying” and “purified” refer to a bacterium or other material that has been separated from at least some of the components with which it was associated either when initially produced or generated (e.g., whether in nature or in an experimental setting), or during any time after its initial production. A bacterium or a bacterial population may be considered purified if it is isolated at or after production, such as from a material or environment containing the bacterium or bacterial population, and a purified bacterium or bacterial population may contain other materials up to about 10%, about 20%, about 30%, about 40%, about 50%, about 60%, about 70%, about 80%, about 90%, or any range derivable therein, or above about 90% and still be considered “isolated.” In some aspects, purified bacteria and bacterial populations are more than about 80%, about 85%, about 90%, about 91%, about 92%, about 93%, about 94%, about 95%, about 96%, about 97%, about 98%, about 99%, or any range derivable therein, or more than about 99% pure. In the instance of bacterial compositions provided herein, the one or more bacterial types present in the composition can be independently purified from one or more other bacteria produced and/or present in the material or environment containing the bacterial type. Bacterial compositions and the bacterial components thereof are generally purified from residual habitat products.

An effective amount of the pharmaceutical composition is determined based on the intended goal. The term “unit dose” or “unit dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

It is contemplated that any aspect discussed in this specification can be implemented with respect to any method or composition of the disclosure, and vice versa. Furthermore, compositions of the disclosure can be used to achieve methods of the disclosure.

### II. Bacteroides ovatus

Allo-HSCT is a curative therapy for high-risk hematological malignancies, but complications such as infections and GVHD continue to limit its success. The intestinal microbiota is an important modulator of GVHD, and broad-spectrum antibiotics are known to increase the incidence of aGI-GVHD by compromising several functions of an intact intestinal microbiota, resulting in alterations to the intestinal environment including reduced concentrations of metabolic products in the colonic lumen (Hayase et al., 2022). The poor prognosis of severe aGI-GVHD underlines the need to better understand how intestinal microbes can help suppress GVHD in allo-HSCT.

In aspects herein, the inventors investigated the impact of the intestinal microbiota on treatment responsiveness of aGI-GVHD using clinical microbiome data. In a retrospective analysis of the fecal microbiome in aGI-GVHD patients, the inventors found that an altered microbiome profile at presentation of aGI-GVHD and a history of treatment with carbapenem-class antibiotics such as meropenem were significantly associated with developing steroid-refractory GVHD, whereas a high abundance of the commensal species B. ovatus, commonly found in normal individuals, was significantly associated with improved GVHD response to steroid therapy. Consistent with this result, B. ovatus has previously been negatively associated with GVHD (Golob et al., 2017). However, it has not been well studied whether B. ovatus can mechanistically suppress severe GVHD.

Some prior studies have reported that B. ovatus can mediate multiple beneficial functions in maintaining intestinal homeostasis in the host via production of indole-3-acetic acid or sphingolipid production (Brown et al., 2019; Ihekweazu et al., 2021). Here, in a murine model, the inventors found that introduction of B. ovatus resulted in improved survival in meropenem-treated allo-HSCT mice but not in meropenem-untreated allo-HSCT mice. This suggested that B. ovatus helped suppress GVHD only in hosts with a disrupted microbiota, and that a key function of B. ovatus may be related to mechanisms underlying aggravated colonic GVHD in the setting of antibiotic injury. Unlike B. ovatus, B. theta is known to be capable of utilizing host-derived glycans (Bergstrom and Xia, 2013; Tailford et al., 2015), and was found to aggravate meropenem-induced colonic GVHD in a prior study (Hayase et al., 2022). In this study, the inventors found that in the setting of an antibiotic-disrupted microbiota with expansion of mucus-degrading B. theta, the introduction of B. ovatus ameliorated the severity of colonic GVHD via polysaccharide degradation, thus producing abundant monosaccharides and improving the intestinal metabolomic environment in allo-HSCT.

In summary, an antibiotic-disrupted microbiota caused by carbapenems including meropenem increased the severity of intestinal GVHD and was associated with treatment-refractory aGI-GVHD in patients. Mouse modeling demonstrated that introducing B. ovatus can ameliorate the severity of GVHD in a model of meropenem-induced colonic GVHD. This understanding of how specific bacteria such as B. ovatus can reduce intestinal inflammation should facilitate the development of new strategies to better prevent and treat this important limitation of allo-HSCT. To elucidate how a healthy microbiome improves the treatment responsiveness of GVHD, the inventors still need further studies. In certain aspects, murine GVHD model studies combined with in vitro assays, confirmed that Bacteroides ovatus ameliorated meropenem-induced colonic GVHD via xylose-comprising polysaccharide degradation. Further, Bacteroides ovatus has broad ability not only carbohydrate degradation but also production of tryptophan metabolites (Ihekweazu et al., 2021), sphingolipid (Brown et al., 2019), or bile salt hydrolase (Yoon et al., 2017) and secretion of fecal IgA (Yang et al., 2020).

In further aspects herein, the inventors evaluate the impact of the composition of the intestinal microbiota at the onset of aGI-GVHD on GVHD severity. Retrospective analysis of 37 aGI-GVHD patients showed that steroid-refractory GVHD was significantly associated with higher clinical stages and histological grades of aGI-GVHD at the onset of aGI-GVHD. An examination of the intestinal microbiome collected from aGI-GVHD patients at the onset of aGI-GVHD revealed that steroid-refractory patients showed greater dysbiosis than responsive patients. Furthermore, higher abundances of Bacteroides ovatus were significantly associated with improved response to steroid therapy in aGI-GVHD patients.

The inventors recently found that in a murine GVHD model, treatment with meropenem, a commonly used carbapenem in allo-HSCT patients, expanded a mucus-degrading bacterial species, Bacteroides thetaiotaomicron (B. theta), and aggravated colonic GVHD¹⁶. Using this model, the inventors evaluated for the impact of B. ovatus on GVHD severity in mice with meropenem-aggravated colonic GVHD. Consistent with the clinical findings, the inventors found that introduction of B. ovatus improved survival of mice with meropenem-aggravated colonic GVHD. B. ovatus also inhibited the expansion and functionality of mucus-degrading bacteria including B. theta and Akkermansia muciniphila. Meropenem altered not only the microbiome composition but also the intestinal environment, including the levels of carbohydrates, and altered functions of intestinal microbes due to changes of metabolic substrates in the colonic lumen can strongly modulate GVHD severity¹⁶ B. ovatus has been reported to be non-mucus-degrading bacteria and have a broad spectrum of polysaccharide-degrading functions including metabolizing xylose-comprising polysaccharides such as xylan¹⁷. Importantly, mice treated with a mutant strain of B. ovatus unable to degrade xylan demonstrated worse survival compared to those treated with wild-type B. ovatus. The ability of B. ovatus to degrade xylose-comprising polysaccharides and produce abundant monosaccharides including xylose in the colonic lumen may play a key role in improving the intestinal metabolic environment in allo-HSCT and prevent expansion of mucus-degrading bacteria, leading to favorable outcomes of aGI-GVHD.

### III. Therapeutic Compositions

Certain aspects relate to therapeutic compositions comprising a bacteria. The bacteria may be a strain of Bacteroides ovatus. In some aspects, the therapeutic composition comprises, consists of, or consists essentially of a bacteria, including a Bacteroides ovatus, in a unit dosage. The unit dosage may be any dosage sufficient for the desired effect of the therapeutic composition. In some aspects the unit dosage comprises between 1×10³ to 9×10¹⁶ colony forming units (CFU) of the bacteria. In some aspects the unit dosage comprises at least, at most, or about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, or any range derivable therein, CFU of the bacteria. In another aspect, the disclosure relates to compositions comprising an isolated or purified population of Bacteroides ovatus. Therapeutic compositions and methods of administering Bacteroides ovatus may involve such unit dosages. Moreover, a unit dosage may be given multiple times over a time period as discussed below.

Certain aspects relate to therapeutic compositions comprising a polysaccharide. The polysaccharide may be any polysaccharide, including a dietary-derived polysaccharide. In certain aspects, the polysaccharide comprises a xylose or xylose analog. In certain aspects, the polysaccharide comprises arabinoxylan, including cereal grain arabinoxylan, such as wheat arabinoxylan, and/or xyloglucan, including XXGG-type and/or a XXXG-type xyloglugan, such as tamarind xyloglucan.

In some aspects, the therapeutic composition does not comprise a certain bacteria. In some aspects, the therapeutic composition does not comprise a Bacteroides thetaiotaomicron.

The therapeutic compositions can be formulated for administration, including as pharmaceutical formulations, e.g., formulated for oral administration; suppository administration; or injection such as via the intravenous, intramuscular, subcutaneous, or intraperitoneal routes. Such compositions can be prepared as either liquid solutions or suspensions; solid forms suitable for use to prepare solutions or suspensions upon the addition of a liquid prior to injection can also be prepared; and, the preparations can also be emulsified.

In certain aspects, the therapeutic composition, which may include Bacteroides ovatus and optionally a polysaccharide, is formulated for oral administration. The formulation for oral administration may comprise a pill, capsule, suspension, drink, or the like. In some aspects, the polysaccharide is administered through food.

In some aspects, the therapeutic composition comprising Bacteroides ovatus is a fecal transplant. In some aspects, the fecal matter is administered in a dose of 50 g. In some embodiments, the fecal matter is administered in a dose of at least, at most, or exactly 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 325, 350, 375, or 400 g (or any derivable range therein). In certain aspects, the fecal transplant comprises fecal matter collected from a patient that has not received a stem cell therapy or antibiotic in 1, 2, 3, 4, 5, 6, 7, 8, 9, 10 days, weeks, months, and/or years (and any range derivable therein) prior to collecting the fecal matter. In certain aspects, the fecal transplant comprises fecal matter that comprises a measurable amount of Bacteroides ovatus. In certain aspects, the fecal transplant comprises fecal matter that comprises a therapeutically effective amount of Bacteroides ovatus.

The pharmaceutical formulations suitable for injectable use include sterile aqueous solutions or dispersions; formulations including, for example, aqueous propylene glycol; and sterile powders for the extemporaneous preparation of sterile injectable solutions or dispersions. In certain aspects, the formulation is stable under the conditions of manufacture and storage and preserved against the contaminating action of non-therapeutic microorganisms.

A pharmaceutical composition or formulation can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of unintended microorganisms can be brought about by various anti-bacterial and anti-fungal agents, for example, parabens, chlorobutanol, phenol, sorbic acid, thimerosal, and the like. In certain aspects, the formulation includes isotonic agents, for example, sugars or sodium chloride. Prolonged absorption of the injectable compositions can be brought about by the use in the compositions of agents delaying absorption, for example, aluminum monostearate and gelatin.

Injectable solutions may be prepared by incorporating the active compounds in the required amount in the appropriate solvent with various other ingredients enumerated above, as required. In certain aspects encompassing powders for the preparation of injectable solutions, the therapeutic composition(s) are vacuum-dried and/or freeze-dried, which yield a powder of the active ingredient, plus any additional desired ingredient.

The present disclosure also provides a pharmaceutical composition comprising one or more microbial cultures as described above. The bacterial species therefore are present in the dose form as live bacteria, whether in dried, lyophilized, or sporulated form. This may be preferably adapted for suitable administration; for example, in tablet or powder form, potentially with an enteric coating, for oral treatment.

In particular aspects, the composition is formulated for oral administration. Oral administration may be achieved using a chewable formulation, a dissolving formulation, an encapsulated/coated formulation, a multi-layered lozenge (to separate active ingredients and/or active ingredients and excipients), a slow release/timed release formulation, or other suitable formulations known to persons skilled in the art. Although the word “tablet” is used herein, the formulation may take a variety of physical forms that may commonly be referred to by other terms, such as lozenge, pill, capsule, or the like.

While the compositions of the present disclosure are preferably formulated for oral administration, other routes of administration can be employed, however, including, but not limited to, intracolonic, subcutaneous, intramuscular, intradermal, transdermal, intraocular, intraperitoneal, mucosal, vaginal, rectal, and intravenous.

In another aspect, the disclosed composition may be prepared as a suppository. The suppository may include but is not limited to the bacteria and one or more carriers, such as polyethylene glycol, acacia, acetylated monoglycerides, carnuba wax, cellulose acetate phthalate, corn starch, dibutyl phthalate, docusate sodium, gelatin, glycerin, iron oxides, kaolin, lactose, magnesium stearate, methyl paraben, pharmaceutical glaze, povidone, propyl paraben, sodium benzoate, sorbitan monoleate, sucrose talc, titanium dioxide, white wax and coloring agents.

In some aspects, the composition may be prepared as a tablet. The tablet may include the bacteria and one or more tableting agents (i.e., carriers), such as dibasic calcium phosphate, stearic acid, croscarmellose, silica, cellulose and cellulose coating. The tablets may be formed using a direct compression process, though those skilled in the art will appreciate that various techniques may be used to form the tablets.

In other aspects, the composition may be formed as food or drink or, alternatively, as an additive to food or drink, wherein an appropriate quantity of bacteria is added to the food or drink to render the food or drink the carrier.

The compositions of the present disclosure may further comprise one or more prebiotics known in the art, such as lactitol, inulin, or a combination thereof.

In some aspects, the composition may further comprise a food or a nutritional supplement effective to stimulate the growth of Bacteroides ovatus present in the gastrointestinal tract of the subject. In some aspects, the nutritional supplement is produced by a bacterium associated with a healthy human gut microbiome.

### IV. Administration of Therapeutic Compositions

Certain aspects concern the administration of therapies and therapeutic compositions, including any therapeutic composition described herein that includes Bacteroides ovatus. The therapies may be administered in any suitable manner known in the art. The therapy provided herein may comprise administration of a combination of therapeutic composition, such as a first composition and a second composition. In some aspects, the first composition comprises any Bacteroides ovatus comprising composition described herein. In some aspects, the second composition comprises a polysaccharide, including any polysaccharide described herein. The first and second compositions may be administered sequentially (at different times) or simultaneously (at the same time). In some aspects, the first and second compositions are administered as separate compositions. In some aspects, the first and second compositions are administered as the same composition.

In some aspects, the first therapeutic composition and the second therapeutic composition are administered substantially simultaneously. In some aspects, the first therapeutic composition and the second therapeutic composition are administered sequentially. In some aspects, the first therapeutic composition, the second therapeutic composition, and a third therapy, which may be a stem cell therapy and/or an antibiotic, are administered sequentially or simultaneously. In some aspects, the first and second therapeutic compositions are administered concurrently and the third therapy is administered sequentially, before and/or after, with the first and second therapeutic compositions. In some aspects, the first therapeutic composition is administered before administering the second therapeutic composition. In some aspects, the first therapeutic composition is administered after administering the second therapeutic composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

In some aspects, the first composition, which may be a Bacteroides ovatus composition, and optionally the second composition, which may be a polysaccharide composition, are administered prophylactically. The first composition and optionally the second composition may be administered before the administration of a treatment that causes a disease or disorder. In some aspects, the first composition and optionally the second composition are administered before the administration of a stem cell therapy (including any stem cell transplantation described herein) and/or before the administration of an antibiotic (including any antibiotic described herein). The first composition and optionally the second composition may be administered prophylactically, as described herein, at any time before the administration of the stem cell therapy and/or antibiotic, including 1, 2, 3, 4, 5, 6, 7, 8, 9, 10, 11, 12, or more, hours, days, weeks, and/or months (or any range derivable therein) before the administration of the stem cell therapy and/or antibiotic.

In some aspects, the first composition, which may be a Bacteroides ovatus composition, and optionally the second composition, which may be a polysaccharide composition, are administered substantially simultaneously with another composition. The first composition and optionally the second composition may be administered substantially simultaneously with the administration of a treatment that causes a disease or disorder. In some aspects, the first composition and optionally the second composition are administered substantially simultaneously with the administration of a stem cell therapy (including any stem cell transplantation described herein) and/or before the administration of an antibiotic (including any antibiotic described herein). In certain aspects, the stem cell therapy and/or antibiotic is administered over a time period, which may be 1, 2, 3, 4, 5, 6, 7, 8, 9, 10, 11, 12, or more, hours, or more days, weeks, months, and/or years (and any range derivable therein). In such aspects, the first composition and optionally the second composition may be administered over all or part of the time period.

In some aspects, the first composition, which may be a Bacteroides ovatus composition, and optionally the second composition, which may be a polysaccharide composition, are administered after the administration of another composition. The first composition and optionally the second composition may be administered after the administration of a treatment that causes a disease or disorder. In some aspects, the first composition and optionally the second composition are administered after the administration of a stem cell therapy (including any stem cell transplantation described herein) and/or before the administration of an antibiotic (including any antibiotic described herein). The first composition and optionally the second composition may be administered at any time after the administration of the stem cell therapy and/or antibiotic, including 1, 2, 3, 4, 5, 6, 7, 8, 9, 10, 11, 12, or more, hours, days, weeks, and/or months (and any range derivable therein) after the administration of the stem cell therapy and/or antibiotic.

In certain aspects, the first composition and optionally the second composition—administered before, simultaneously with, or after a treatment that causes a disease or disorder—is administered in an amount that prevents, reduces the severity of, or treats the disease or disorder. In certain aspects, the first composition and optionally the second composition—administered before, simultaneously with, or after a stem cell therapy and/or antibiotic—is administered in an amount that prevents, reduces the severity of, or treats the disease or disorder caused by the stem cell therapy and/or antibiotic. Such amount may be referred to herein as a therapeutically effective amount.

The therapeutic agents of the disclosure may be administered by the same route of administration or by different routes of administration. In some aspects, the therapeutic composition is administered intracolonically, intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. In some aspects, the composition is administered via gavage. The appropriate dosage may be determined based on the type of disease to be treated, severity and course of the disease, the clinical condition of the patient, the patient's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, a single dose of the first therapeutic composition, which comprises Bacteroides ovatus, is administered. In some aspects, multiple doses of the first therapeutic composition are administered. In some aspects, the first therapeutic composition is administered at a dose of between 1×10⁵ to 9×10⁸ CFUs, or any range derivable therein. In some aspects, the first therapeutic composition is administered at a dose of at least, at most, or about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, or any range derivable therein, CFU of the bacteria.

In some aspects, a single dose of the second therapeutic composition, which may comprise a polysaccharide, is administered. In some aspects, multiple doses of the second therapeutic composition are administered. In some aspects, the second therapeutic composition is administered at a dose of 1 μg/kg to 1 mg/kg, or any range derivable therein, or between 1 mg/kg and 100 mg/kg, or any range derivable therein. In some aspects, the second therapeutic composition is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein, μg/kg or mg/kg.

The quantity to be administered, both according to number of treatments and unit dose, depends on the treatment effect desired. An effective dose is understood to refer to an amount necessary to achieve a particular effect.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more (and any range derivable therein) administrations. The administrations can be at 1, 2, 3, 4, 5, 6, 7, 8, to 5, 6, 7, 8, 9, 10, 11, 12, 13, 14 day, week, or month intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

Administration of the compositions will typically be via any common route. This includes, but is not limited to oral, suppository, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

The desired dose of the composition of the present disclosure may be presented in multiple (e.g., two, three, four, five, six, or more) sub-doses administered at appropriate intervals throughout the day.

Upon formulation, solutions will be administered in a manner compatible with the dosage formulation and in such amount as is therapeutically or prophylactically effective. The formulations are easily administered in a variety of dosage forms, such as the type of injectable solutions described above.

### V. Methods of Treatment

Provided herein are methods for treating or delaying progression of certain diseases or disorders by administration of therapeutic compositions, such as compositions comprising bacteria, including Bacteroides ovatus, to a patient. In some aspects, the patient has been administered, is currently being administered, or will be administered a stem cell therapy and/or an antibiotic. In certain aspects, the disease or disorder comprises an intestinal disorder. The intestinal disorder may comprise intestinal inflammation and/or antibiotic-mediated injury. The disease may comprise a graft versus host disease (GVHD), including acute intestinal graft versus host disease. In some aspects, the patient, who may be have GVHD, is steroid refractory. In some aspects, the patient is steroid refractory when there is no improvement or worsening in at least one symptom or no change in quality of life when the patient is administered a steroid.

In some aspects, the Bacteroides ovatus treatment results in a sustained response, such as a sustained microbiome, in the patient after cessation of the treatment. In some aspects, the Bacteroides ovatus treatment results in prevention or reversal of graft versus host disease.

The term “treatment” or “treating” means any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. In some aspects, the treatment may exclude prevention of the disease.

In certain aspects, the patient administered a therapeutic composition is identified as having, or is at risk of having, a disease, or disorder. In certain aspects, the patient administered a therapeutic composition is identified as having, or is at risk of having, a certain response to a treatment. The response to the treatment may be graft versus host disease. The response to the treatment may be that the patient is, or is not, responsive to a steroid therapy, including a steroid therapy administered before, during, and/or after administration of a stem cell therapy. The responsiveness to the steroid therapy may refer to the patient given a stem cell therapy being responsive or refractive to a steroid administration. The steroid therapy may be any steroid therapy administered to a patient, including prednisone, prednisolone, dexamethasone, and/or hydrocortisone.

In certain aspects, the administration of a therapeutic composition to a patient alters the microbiome, including altering the bacteria in the microbiome, and/or microbiome environment in the patient. In some aspects, the administration of the therapeutic composition decreases the amount of at least one bacteria and/or genus of bacteria. In some aspects, the administration of the therapeutic composition decreases the amount of Bacteroides thetaiotaomicron. In some aspects, the administration of the therapeutic composition alters the microbiome by altering the expression of at least one gene expressed in bacteria of the microbiome. The gene(s) may comprise a polysaccharide utilization loci (PUL) gene. In some aspects, the administration of the therapeutic compositions to a patient alters, including by downregulation, one or more PULs in a bacteria, including Bacteroides thetaiotaomicron, present in the microbiome of the patient. The PULs may contribute to the degradation of mucin O-glycans. In some aspects, the PULs are PUL12, PUL14, PUL16, and/or PUL78.

### VI. Methods of Determining Microbiome Composition

In some aspects, the methods relate to obtaining a microbiome profile of a patient. In some aspects, obtaining a microbiome profile comprises the steps of or the ordered steps of: i) obtaining a sample obtained from a subject (e.g., a human subject), ii) isolating one or more bacterial species from the sample, iii) isolating one or more nucleic acids from at least one bacterial species, iv) sequencing the isolated nucleic acids, and v) comparing the sequenced nucleic acids to a reference nucleic acid sequence. When performing the methods necessitating genotyping, any genotyping assay can be used. For example, this can be done by sequencing the 16S or the 23S ribosomal subunit or by metagenomics shotgun DNA sequencing associated with metatranscriptomics.

In some aspects, obtaining the microbiome profile of a patient is used to monitor the need of administering the therapeutic compositions described herein to the patient. In certain aspects, obtaining the microbiome profile of a patient is used to monitor the efficacy of the therapeutic compositions administered to the patient, including monitoring the concentration of Bacteroides ovatus in the microbiome profile. In certain aspects, the patient is or is not administered a therapeutic composition based on the obtained microbiome profile of the patient. In certain aspects, the patient is administered a therapeutic composition because the obtained microbiome profile is more or less diverse when compared to a standard. Diversity may be compared by any method known in the art, including the weighted-UniFrac method. In certain aspects, the patient is administered a therapeutic composition because the obtained microbiome profile has an increased and/or decreased amount of one or more bacteria species and/or genus of bacteria when compared to a standard. In some aspects, the microbiome profile has an increased amount of Enterococcus, Citrobacter, Streptococcus, Staphylococcus, and/or Enterobacter bacteria when compared to a standard. In some aspects, the microbiome profile has a decreased amount of Bacteroides, Prevotella, Faecalibacterium, and/or UBA1819 bacteria when compared to a standard.

In certain aspects, the standard for comparison of the microbiome profile is a microbiome profile from a healthy individual. The healthy individual may be a patient that has not received a stem cell therapy and/or antibiotic in 1, 2, 3, 4, 5, 6, 7, 8, 9, 10, 11, 12 13, 14, or more days, weeks, months, and/or years (and any range derivable therein). In certain aspects, the healthy individual is a patient that does not have a diagnosed intestinal disorder.

Methods for determining microbiome composition may include one or more microbiology methods such as sequencing, next generation sequencing, wester blotting, comparative genomic hybridization, PCR, ELISA, etc.

In some aspects, the patient receiving a therapeutic composition, including any therapeutic composition described herein, has a higher abundance of at least one bacteria species and/or genus of bacteria. In certain aspects, the patient having, or suspected of having, GVHD, including acute gastrointestinal GVHD, has a higher abundance of at least one bacteria species and/or genus of bacteria. In some aspects, the patient who is, or is suspected of being steroid-refractive, has a higher abundance of at least one bacteria species and/or genus of bacteria. The bacteria genus may be Enterococcus.

Certain aspects relate to methods of prognosing the effectiveness of a therapy in a patient. In some aspects, the method prognoses the effectiveness of a steroid therapy in a patient, i.e. determining whether the patient is steroid refractory. The patient may have received, or will receive, a stem cell therapy and/or antibiotic. In certain aspects, the effectiveness of the therapy is determined by obtaining a microbiome profile from the patient. In some aspects, the therapy, including a steroid therapy, is determined to be ineffective when the microbiome profile is different, which may be less diverse, from a standard microbiome profile. In some aspects, the therapy, including a steroid therapy, is determined to be ineffective when the microbiome profile comprises a lower abundance of Bacteroides, Prevotella, Faecalibacterium, and/or UBA1819 compared to a standard. In some aspects, the therapy, including a steroid therapy, is determined to be ineffective when the microbiome profile comprises higher abundances of the genera Citrobacter, Streptococcus, Staphylococcus, and/or Enterobacter compared to a standard. The standard may be a microbiome profile from a healthy individual. The healthy individual may be a patient that has not received a stem cell therapy and/or antibiotic in 1, 2, 3, 4, 5, 6, 7, 8, 9, 10, 11, 12 13, 14, or more days, weeks, months, and/or years (and any range derivable therein). The standard may be a microbiome profile from a patient that was responsive to a steroid therapy.

### VII. Kits

Certain aspects of the disclosure also encompass kits for performing the methods of the disclosure. Such kits can be prepared from readily available materials and reagents. For example, such kits can comprise any one or more of the following materials: enzymes, reaction tubes, buffers, detergent, primers, probes, antibodies.

In a particular aspect, these kits may comprise a plurality of agents for assessing or identifying microorganisms, wherein the kit is housed in a container. The kits may further comprise instructions for using the kit for assessing sequences, means for converting and/or analyzing sequence data to generate prognosis.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

Further kit aspects relate to kits comprising the therapeutic compositions of the disclosure. The kits may be useful in the treatment methods of the disclosure and comprise instructions for use.

### VIII. Bacteria Isolation and Culture

In some aspects, bacteria, including Bacteroides ovatus, are isolated and/or purified from a source. The bacteria may be isolated and/or purified from any suitable source. In some aspects, the source is a human sample. In some aspects, the source is a non-human sample, such as a rodent sample. Bacteria, including Bacteroides ovatus, may be isolated and/or purified using any method known in the art. The isolated and/or purified bacteria may then be formulated into therapeutic compositions, including the therapeutic compositions described herein. Before or after being isolated and/or purified, the bacteria may be characterized, including by sequencing to determine the composition and identity of the isolated and/or purified bacteria as Bacteroides ovatus. The bacteria may be characterized by 16S rRNA or 23S rRNA sequencing.

In certain aspects, the methods described herein of treating a patient and methods comprising administering Bacteroides ovatus to a patient further comprise isolating and/or purifying the Bacteroides ovatus prior to the treatment and/or administration. The purified and/or isolated Bacteroides ovatus may be used in the methods described herein.

In some aspects, the isolated and/or purified bacteria may be cultured for at least between about 1 days and about 40 days, for at least between about 5 days and about 35 days, for at least between about 5 days and 21 days. The bacteria may be cultured to generate sufficient quantity of bacteria to reach a unit dosage. The bacteria may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or any range derivable therein, or more days. The bacteria may be cultured in the presence of a liquid culture medium, such as an LB broth or other suitable medium for culturing bacteria. Typically, the medium may comprise a basal medium formulation as known in the art. Compositions of the above basal media are generally known in the art, and it is within the skill of one in the art to modify or modulate concentrations of media and/or media supplements as necessary for the bacteria cultured. A defined medium, however, also can be used if the growth factors, cytokines, and hormones necessary for culturing the bacteria are provided at appropriate concentrations in the medium. Media useful in the methods of the disclosure may comprise one or more compounds of interest, including, but not limited to, antibiotics, mitogenic compounds, or differentiation compounds useful for the culturing of bacteria. The bacteria may be grown at temperatures between 27° C. to 40° C., such as 31° C. to 37° C., or any range derivable therein, and may be in a humidified incubator.

### IX. Additional Therapies

It is contemplated that other additional therapies may be used in combination with certain aspects of the present therapeutic compositions to improve the therapeutic efficacy of treatment. In some aspects, the additional therapy comprises an additional bacteria or bacteria strain that is not Bacteroides ovatus. The additional bacteria or bacteria strains may comprise bacteria found in the microbiome of a patient, administered a stem cell therapy and/or antibiotic, that do not develop an intestinal disease, including any intestinal disease described herein, caused by the stem cell therapy and/or antibiotic. In certain aspects, the additional therapy comprises a therapeutic composition capable of treating, including preventing or reducing, GVHD, including for example, immunomodulating compositions, steroids, antibodies, or other GVHD therapies known in the art.

### EXAMPLES

The following examples are included to demonstrate preferred aspects of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the disclosure.

### Example 1

### Bacteroides-Enriched Microbiome in Allo-HSCT Patients was Associated with a Favorable aGI-GVHD Treatment Response

To investigate the potential impact of intestinal microbiome composition on aGI-GVHD severity and treatment response, the inventors prospectively collected samples from patients at MD Anderson Cancer Center who developed aGI-GVHD in the setting of allo-HSCT from 2017 to 2019. A total of 37 enrolled patients were diagnosed with aGI-GVHD (Table 1): 28 with classic aGI-GVHD and 9 with late-onset aGI-GVHD, by National Institutes of Health consensus criteria¹⁸. The inventors determined treatment response as previously reported¹⁹. All patients received initial therapy with methylprednisolone or prednisone at 2 mg/kg/day followed by tapering per institutional guidelines.

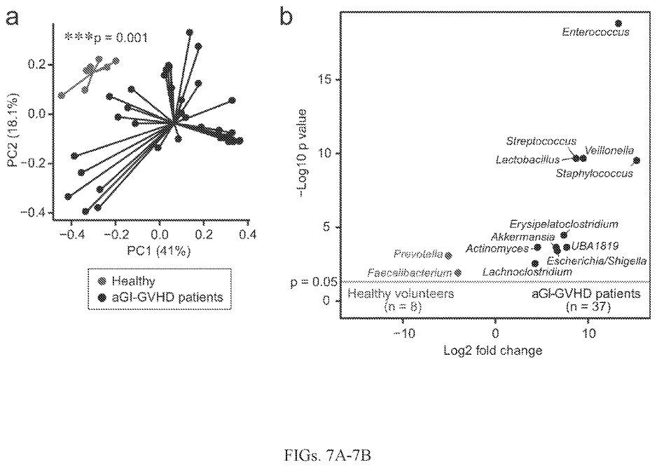

An examination of the microbiome composition of the stool samples using 16S rRNA gene sequencing revealed that the aGI-GVHD cohort showed a significantly distinct intestinal microbiome at the onset of aGI-GVHD from that of healthy volunteers, visualized with principal coordinates analysis (PCoA) and tested using permutational multivariate analysis of variance (PERMANOVA) (FIG. 7A). In particular, aGI-GVHD patients showed significantly higher abundances of the genera Escherichia/Shigella and Enterococcus and reductions in the genera Prevotella and Faecalibacterium (FIG. 7B). These results were consistent with previous reports identifying Escherichia coli and the genus Enterococcus as bacteria that can aggravate GVHD severity^{20,21}.

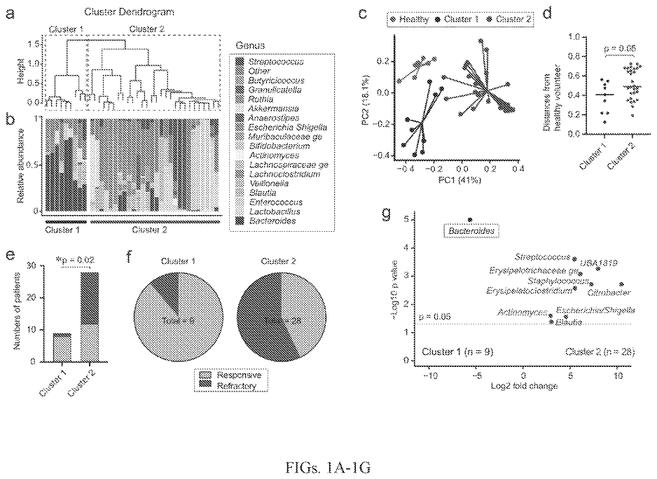

The inventors next sought to identify naturally-occurring subsets within the cohort of aGI-GVHD patients based on differences in microbiome composition. Using hierarchical clustering of weighted UniFrac beta diversity measures, the inventors identified 2 distinct groups, with 9 patients in cluster 1 and 28 patients in cluster 2 (FIGS. 1A, 1B). Other than gender (cluster 1 included a significantly higher proportion of male patients; p=0.01; Table 2), no clinical transplant characteristics were significantly different between clusters 1 and 2. Cluster 1 showed a trend towards less dysbiosis, as measured by weighted UniFrac differences from the microbiome of healthy volunteers (p=0.05; FIGS. 1C, 1D). Interestingly, the inventors found that cluster 1 included a significantly higher proportion of steroid-responsive GVHD patients than cluster 2 (FIGS. 1E, 1F). Performing differential abundance analysis on clusters 1 and 2, the inventors found that cluster 1 was primarily characterized by increased abundance of the genus Bacteroides (FIG. 1G). Overall, these findings suggested that the composition of the intestinal microbiome may be associated with treatment response in patients with aGI-GVHD.

The inventors then investigated whether the composition of the intestinal microbiome at the onset of aGI-GVHD was different between patients who would later be steroid-responsive or steroid-refractory. The aGI-GVHD cohort included 20 patients who responded to steroids and 17 patients who did not. Other than age (refractory patients were significantly younger; p=0.0002; Table 3), no clinical transplant characteristics were significantly different between responsive and refractory patients. The time from allo-HSCT until onset of aGI-GVHD was a median of 31.5 days (range, 14-367 days) in steroid-responsive patients and 42 days (13-257 days) in steroid-refractory patients.

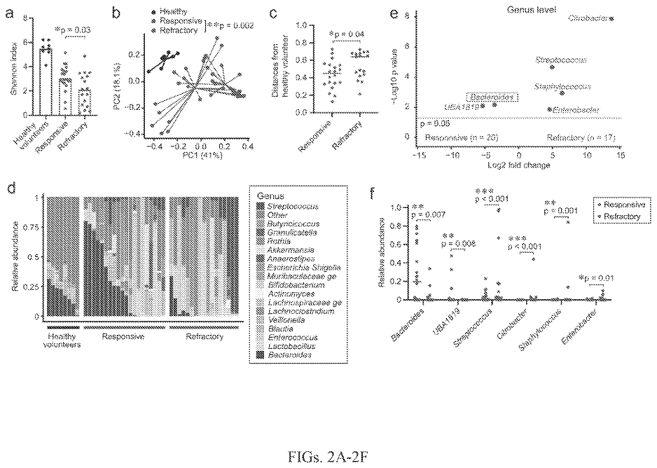

The inventors found that steroid-responsive patients showed significantly higher microbial alpha diversity than steroid-refractory patients, but that this diversity was still lower than that of healthy volunteers (FIG. 2A). Using PCoA with PERMANOVA testing, the inventors found that the intestinal microbiome composition was significantly different between steroid-responsive and steroid-refractory patients (FIG. 2b) and that steroid-refractory patients showed greater dysbiosis than responsive patients, as measured by their weighted UniFrac differences from the microbiome of healthy volunteers (FIGS. 2B, 2C). The inventors evaluated the bacterial taxa that were differentially abundant and found that steroid-refractory patients had reductions in the genera Bacteroides and UBA1819 and higher abundances of the genera Citrobacter, Streptococcus, Staphylococcus, and Enterobacter (FIGS. 2D, 2F).

Overall, these results suggested that alterations of the composition of the intestinal microbiome at clinical presentation of aGI-GVHD were associated with poor response to therapy, and reductions or absence of Bacteroides may contribute to increased aGI-GVHD severity and treatment failure.

### Example 2

### Higher Abundance of Bacteroides Ovatus was Significantly Associated with Favorable aGI-GVHD Treatment Outcomes

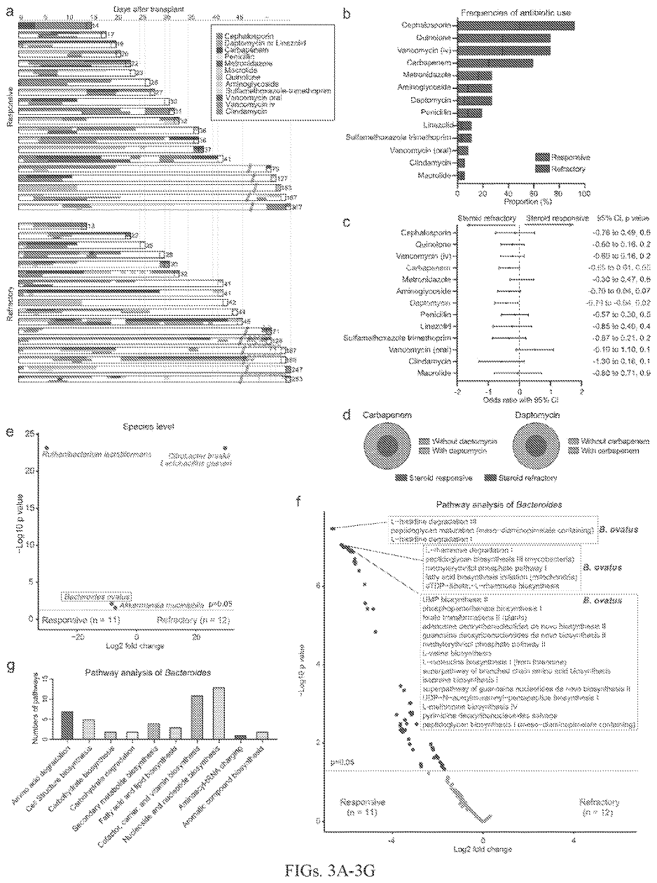

Allo-HSCT patients are often treated with broad-spectrum antibiotics for febrile neutropenia and other infections that arise before as well as after hematopoietic engraftment. These antibiotics, however, can cause bystander damage to intestinal commensals that are critical for maintaining intestinal homeostasis. Indeed, exposure to broad-spectrum antibiotics such as carbapenems has been linked to an increased incidence of aGI-GVHD⁹⁻¹². The inventors examined patient antibiotic treatment histories during the period from allo-HSCT to the onset of aGI-GVHD and looked for associations between treatment with various antibiotic classes and steroid response for GVHD (FIG. 3A). In our institution, quinolone, cephalosporine, carbapenem and intravenous vancomycin were frequently used in our cohort (FIG. 3B). Univariate logistic analysis demonstrated that daptomycin exposure during the period from allo-HSCT was significantly associated with steroid-refractory GVHD and carbapenem exposure was trending towards being associated with steroid-refractory GVHD (p=0.05; FIG. 3C). Most patients who were treated with daptomycin were also treated with a carbapenem during allo-HSCT (FIG. 3D). Importantly, exposure to most antibiotics shifted risks in the direction of steroid-refractory GVHD. These results are consistent with the hypothesis that antibiotic-mediated microbiome disruption could lead to reduced response rates of GVHD to therapy.

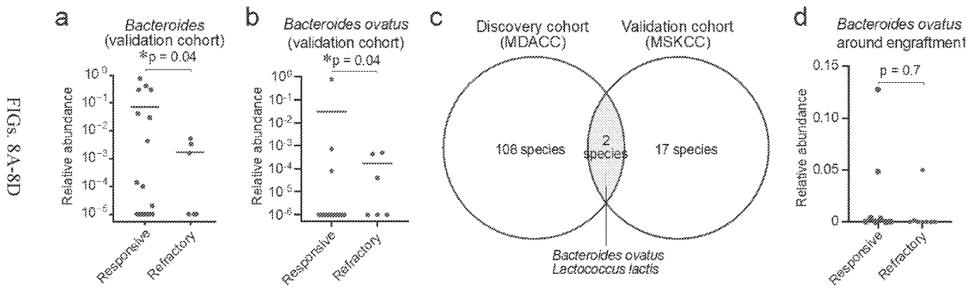

To identify specific species of Bacteroides potentially associated with steroid response for aGI-GVHD, the inventors performed whole-genome sequencing on the subset of fecal samples with sufficient genomic DNA or stool remaining for assessment. In samples from 23 patients, including 11 steroid-responsive patients and 12 steroid-refractory patients, abundances of B. ovatus were significantly increased among the genus Bacteroides in steroid-responsive patients (FIG. 3E). Evaluation of genetic pathways from Bacteroides demonstrated that multiple genetic pathways of Bacteroides were significantly enriched in steroid-responsive patients but none in steroid-refractory patients (FIG. 3F). Interestingly, the top 50 pathways with significantly increased abundances in steroid-responsive patients, including the pathways related to amino acid degradation and carbohydrate biosynthesis/degradation, belonged to B. ovatus (FIG. 3G), indicating that B. ovatus is particularly associated with steroid-responsive GVHD in patients. To validate these results, the inventors additionally investigated the publicly available whole genome sequencing data of 32 fecal samples collected at the onset of aGI-GVHD in another institution, Memorial Sloan Kettering Cancer Center²². This cohort included 26 steroid-responsive GVHD and 6 steroid-refractory GVHD patients (Table 4). Analysis showed significantly higher abundances of the genus Bacteroides and the species Bacteroides ovatus in steroid-responsive patients compared to refractory patients (FIGS. 8A, 8B). Evaluating differentially abundant microbial species between steroid-responsive and refractory patients that were observed in our discovery and validation cohorts, the inventors found that only B. ovatus and Lactococcus lactis were found in both institutions and both species were significantly associated with steroid-responsive GVHD (FIG. 8C).

The inventors also investigated the impact of abundance of B. ovatus in stool samples prospectively collected on day 14 after allo-HSCT in an additional cohort of 89 patients, which included 16 patients who later developed aGI-GVHD. The inventors found that in these aGI-GVHD patients, day 14 abundances of B. ovatus were not significantly different between those who later developed steroid-responsive and refractory GVHD and were quite low across all patients at this early time point after allo-HSCT (FIG. 2D). This suggested that the microbiome composition of samples collected at the onset of GVHD may be more helpful in predicting GVHD severity and treatment response than samples collected far prior to onset of GVHD.

In summary, results of 16S rRNA and whole-genome sequencing of patient fecal samples at the onset of aGI-GVHD implicated a potential beneficial effect of B. ovatus, which the inventors further examined in a murine GVHD model.

### Example 3

### B. ovatus Suppressed Meropenem-Aggravated Colonic GVHD in A Murine GVHD Model

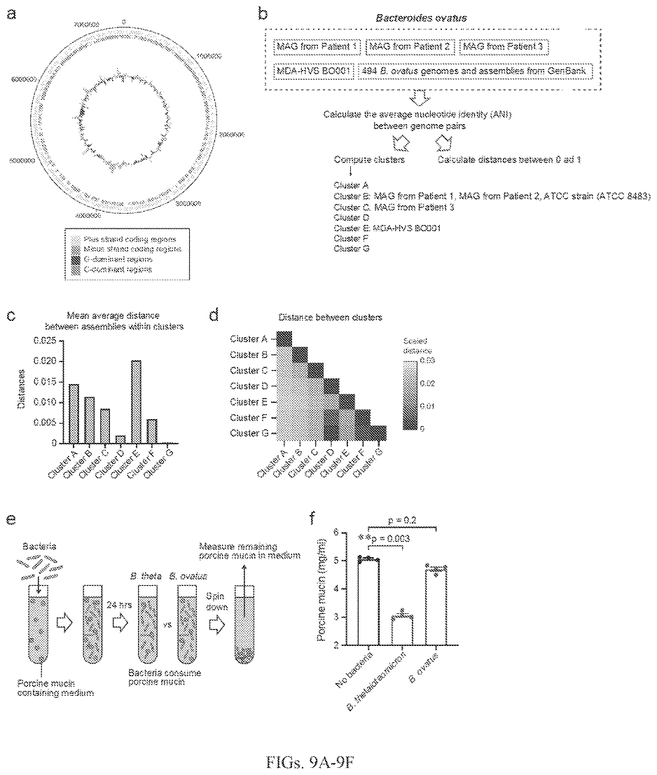

To investigate whether B. ovatus influences GVHD outcomes in a murine GVHD model, the inventors isolated B. ovatus from the stool of a healthy volunteer and named the strain MDA-HVS B0001. The inventors assembled the complete genome of MDA-HVS B0001 and confirmed that it was a strain of B. ovatus, with 99.4% of the genomic identity of the ATCC strain of B. ovatus (ATCC 8483) (FIG. 9A). To quantify the genetic similarity between B. ovatus from aGI-GVHD patients to MDA-HVS B0001 and the ATCC strain of B. ovatus (ATCC 8483), the inventors built metagenome-assembled genomes (MAGs) from sequencing results of 3 responder patient samples with high abundances of B. ovatus (FIG. 9B). The inventors then built a database that included 494 B. ovatus genomes and assemblies available on GenBank that were not tagged as MAG. The inventors calculated the average nucleotide identity (ANI) between genome pairs, calculated both low and high dimensional embeddings, and showed the distances by Uniform Manifold Approximation and Projection (UMAP, FIG. 9B). MDA-HVS B0001 was classified into cluster E whereas the MAGs of 2 patients and the ATCC strain of B. ovatus (ATCC 8483) were classified into cluster B and the other patient-derived MAG into cluster C (FIG. 9B). The mean average distance between assemblies within cluster E was 2.0%, which was large compared to other clusters, indicating that cluster E was a wide cluster (FIG. 9C). The inventors found that the distance between MDA-HVS B0001 and each of the MAGs were 2.5 to 2.6% (FIG. 9D).

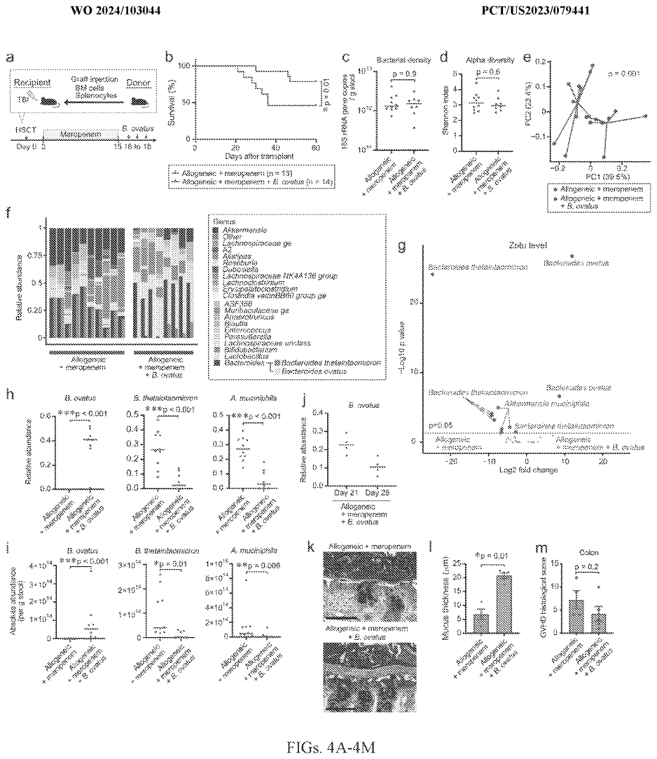

Hereafter, the inventors refer to the isolated B. ovatus, MDA-HVS B0001, as B. ovatus. Because antibiotic-mediated microbiome disruption prior to aGI-GVHD onset could be an important determinant of severe GVHD and a potential risk for the development of steroid-refractory GVHD in allo-HSCT patients (FIG. 3C), the inventors used a previously-described meropenem-aggravated GVHD murine model¹⁶ to evaluate the impact of B. ovatus on GVHD severity. Briefly, lethally-irradiated B6D2F1 (H-2^{b/d}) mice were intravenously injected with 5×10⁶ bone marrow cells and 5×10⁶ splenocytes from major histocompatibility complex (MHC)-mismatched B6 (H-2^{b}) mice on day 0. Meropenem was administered to the allo-HSCT recipient mice in their drinking water on days 3 to 15 relative to allo-HSCT (FIG. 4A). The inventors previously showed that allo-HSCT mice treated with meropenem demonstrated aggravated colonic GVHD in association with loss of the class Clostridia and expansion of B. theta compared to mice not treated with meropenem. B. theta is a species of mucus-degrading bacteria that commonly colonizes the intestinal tract of both mice and humans²³. In this model, expansion of B. theta induces thinning of the colonic mucus layer and increases bacterial translocation, leading to aggravated colonic GVHD.

To compare mucus-degrading functionality between B. ovatus and B. theta, the inventors quantified degradation of mucin-derived carbohydrates in vitro using a periodic acid-Schiff (PAS)-based colorimetric assay (FIG. 9E). As expected, B. theta displayed degradation of mucin-derived carbohydrates, whereas B. ovatus did not (FIG. 9F), suggesting that B. ovatus has less potential to induce mucus-degrading bacteria-related aggravated GVHD, consistent with the prior study²⁴.

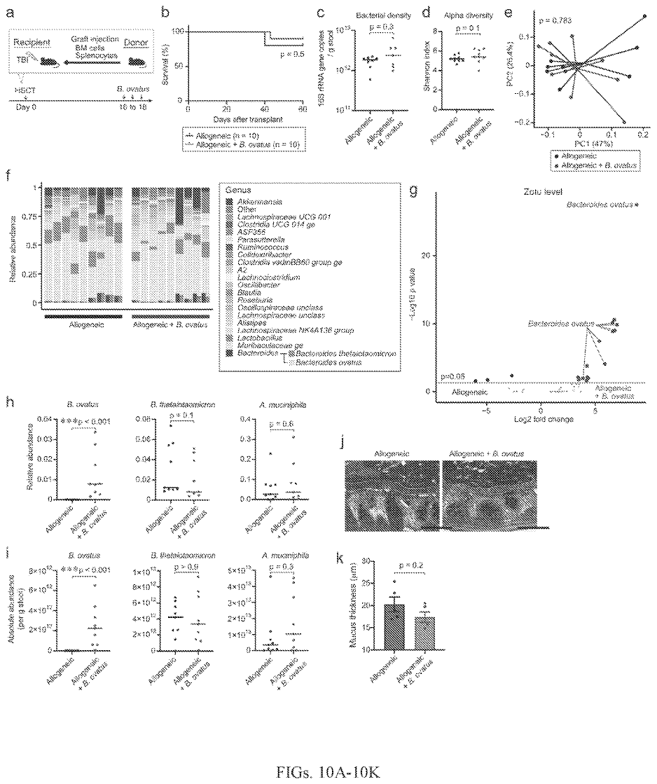

Next, to study the effects of B. ovatus on GVHD severity, the inventors orally inoculated 2×10⁷ colony-forming units of B. ovatus into meropenem-treated allo-HSCT recipient mice daily from days 16 to 18 and monitored GVHD severity and survival (FIG. 4A). Interestingly, the inventors found that meropenem-treated mice that received B. ovatus showed significantly improved survival (FIG. 4B). However, the favorable effects of B. ovatus were not seen in allo-HSCT mice not treated with meropenem (FIGS. 10A, 10B), suggesting that B. ovatus can mitigate the severity of aGI-GVHD only in the context of a disrupted microbiota. This finding, together with the finding that expanded B. theta after meropenem treatment was associated with aggravated colonic GVHD, indicated that different Bacteroides species, which are quite heterogeneous in their metabolic functions, can mediate distinct and even opposing effects on aGI-GVHD^{25,26}. The inventors hypothesized that B. ovatus may mitigate GVHD severity via its metabolic capabilities leading to improved intestinal homeostasis, which was supported by the finding in aGI-GVHD patients that B. ovatus-derived pathways were significantly associated with steroid response (FIG. 3F).

To elucidate potential mechanisms by which B. ovatus mitigated meropenem-aggravated colonic GVHD, the inventors began by examining the microbiota density and composition in both meropenem-treated and untreated allo-HSCT mice with or without introduction of B. ovatus. Introduction of B. ovatus did not alter bacterial density quantified by 16S rRNA gene quantitative polymerase chain reaction (qPCR), or alpha diversity quantified using the Shannon index, in stool collected on day 21 in meropenem-treated mice or in control allo-HSCT mice untreated with meropenem (FIGS. 4C, 4D, 10C, and 10D). Interestingly, the intestinal microbiome composition was significantly altered by administration of B. ovatus in meropenem-treated mice (FIG. 4E). The analysis of differentially abundant bacterial taxa showed reductions in mucus-degrading bacteria such as B. theta and Akkermansia muciniphila in meropenem-treated mice that received B. ovatus in (FIGS. 4F-4I). The inventors also found that colonization by B. ovatus was maintained through day 28 after allo-HSCT (FIG. 4J). Consistent with these results, the thickness of the colonic mucus layer was significantly increased in meropenem-treated mice that received B. ovatus compared to those without B. ovatus (FIGS. 4D, 4L). However, the inventors did not find reduced severity of GVHD histological scores in the colon of meropenem-treated mice treated with B. ovatus (FIG. 4M). On the other hand, meropenem-untreated allo-HSCT mice showed no significant effects of administration of B. ovatus on microbiome composition as a whole, nor on abundances of both B. theta and A. muciniphila (FIGS. 4E-4I) or on colonic mucus layer thickness (FIGS. 10J-10K). These data suggested that B. ovatus suppresses the expansion of mucus-degrading bacteria in mice under certain conditions, such as following meropenem treatment, but does not impact substantially on mucus-degrading bacteria in the absence of prior antibiotics.

### Example 4

### Introducing B. ovatus Suppressed Mucus-Degrading Functionalities by B. theta and A. Muciniphila in a Murine GVHD Model

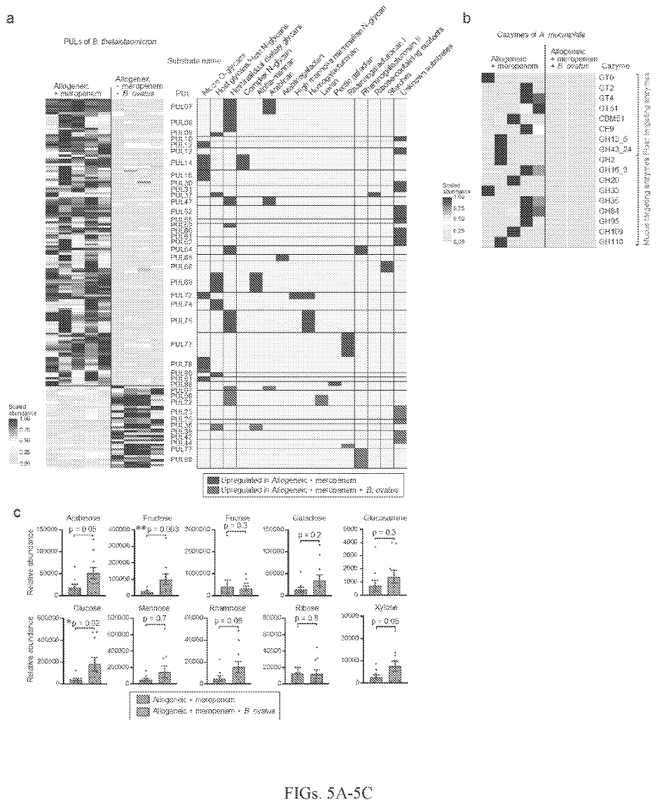

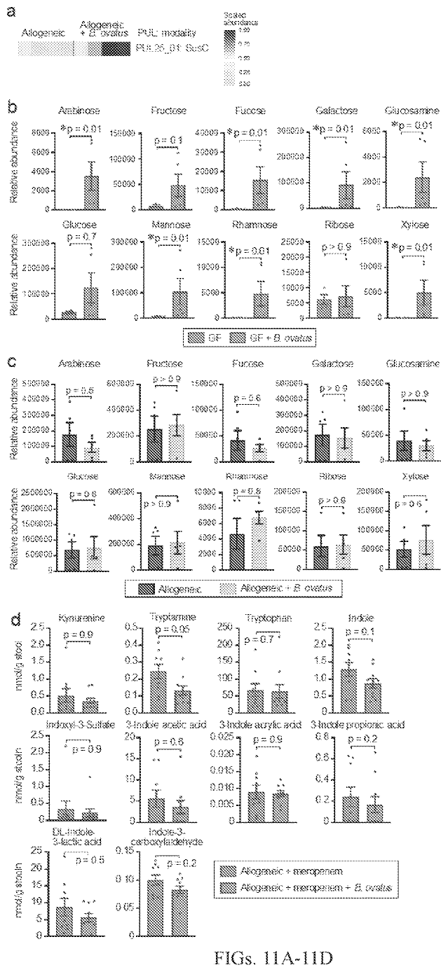

On the basis of the previous finding that meropenem treatment led to changes in carbohydrate levels and mucus-degrading functionalities of B. theta in the murine GVHD model¹⁶, the inventors hypothesized that B. ovatus introduction could be impacting the carbohydrate environment and B. theta gene expression. The inventors began with investigating the effects of B. ovatus on B. theta gene expression in meropenem-treated allo-HSCT mice, by performing microbial RNA sequencing of stool samples. The inventors examined RNA reads from B. theta and annotated these using the polysaccharide utilization loci (PULs) DataBase²⁷. The inventors found that administration of B. ovatus to meropenem-treated allo-HSCT mice led to downregulation in B. theta of many PULs that contribute to degradation of mucin O-glycans (FIGS. 5A, 8). In contrast, in meropenem-untreated mice, administration of B. ovatus did not result in downregulation of any of these PULs by B. theta, which generally displayed very few transcriptomic changes (FIG. 11A), supporting the prior finding that B. theta in meropenem-untreated mice did not upregulate mucus-degrading functionalities.¹⁶.

Since the abundances of A. muciniphila were also suppressed by administration of B. ovatus in meropenem-treated mice (FIGS. 4G-4I), the inventors next investigated the effect of B. ovatus on A. muciniphila gene expression in meropenem-treated allo-HSCT mice. Microbial RNA sequencing data showed that administration of B. ovatus downregulated A. muciniphila genes that contribute to degradation of mucin O-glycans in meropenem-treated mice (FIG. 5B). These results suggested that B. ovatus not only suppressed expansion of mucus-degrading bacteria, but also produced downregulation of mucus-degrading functionalities in B. theta and A. muciniphila in meropenem-treated allo-HSCT mice.

In the previous study of meropenem-aggravated colonic GVHD, the inventors found that mucus-degrading functionalities of B. theta are repressed by higher concentrations of ambient monosaccharides, including especially xylose¹⁶. The inventors thus quantified effects of B. ovatus on colonic luminal concentrations of monosaccharides using ion chromatography-mass spectrometry (IC-MS). Interestingly, most monosaccharides were markedly increased in meropenem-treated mice that received introduction of B. ovatus (FIG. 5C), indicating that B. ovatus may be raising concentrations of monosaccharides by helping to degrade dietary-derived polysaccharides. To evaluate if B. ovatus was sufficient to elevated monosaccharide concentrations by itself without contributions from other intestinal bacteria, the inventors utilized gnotobiotic mouse models. The inventors measured carbohydrate concentrations of colonic luminal contents collected from previously germ-free (GF) mice two weeks after introduction of B. ovatus. The inventors found increased concentrations of many monosaccharides in the colonic lumen of mice monocolonized with B. ovatus, while GF mice had very low concentrations of nearly all monosaccharides except ribose (FIG. 11B). As expected, monosaccharide concentrations in the colonic lumen of meropenem-untreated mice were not significantly affected by B. ovatus introduction (FIG. 11C). These results suggested that B. ovatus functions in the setting of an injured microbiota to elevate concentrations of monosaccharides in the colonic lumen. It has also been reported that B. ovatus can produce indole-3-acetic acid and promote interleukin-22 production from immune cells, leading to decreased colonic inflammation in a murine inflammatory bowel disease model²⁸. The inventors did not observe, however, significant changes in concentrations of tryptophan metabolites due to B. ovatus in the model (FIG. 11D). Thus, the results indicated that B. ovatus is effective in elevating concentrations of monosaccharides in the colonic lumen of mice compared to mice with an absent or injured microbiota.

### Example 5

### Degradation of Xylose-Comprising Polysaccharides by B. ovatus Suppressed Mucus-Degrading Functionalities in B. Theta

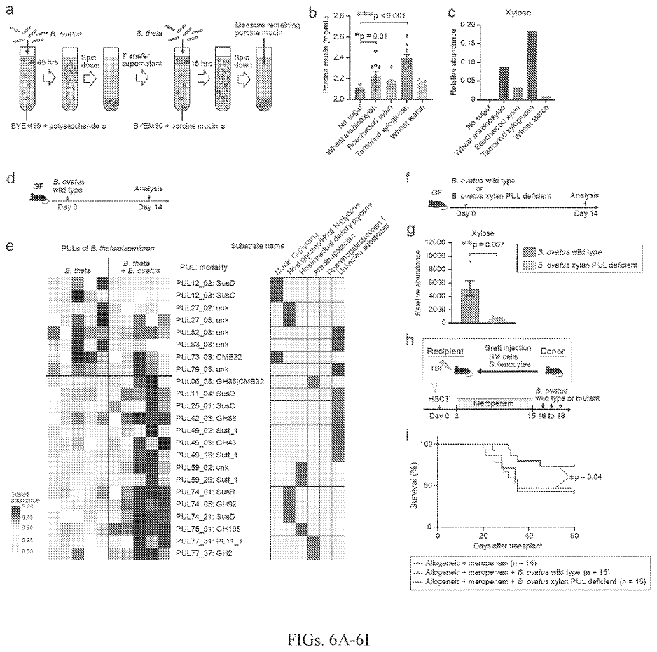

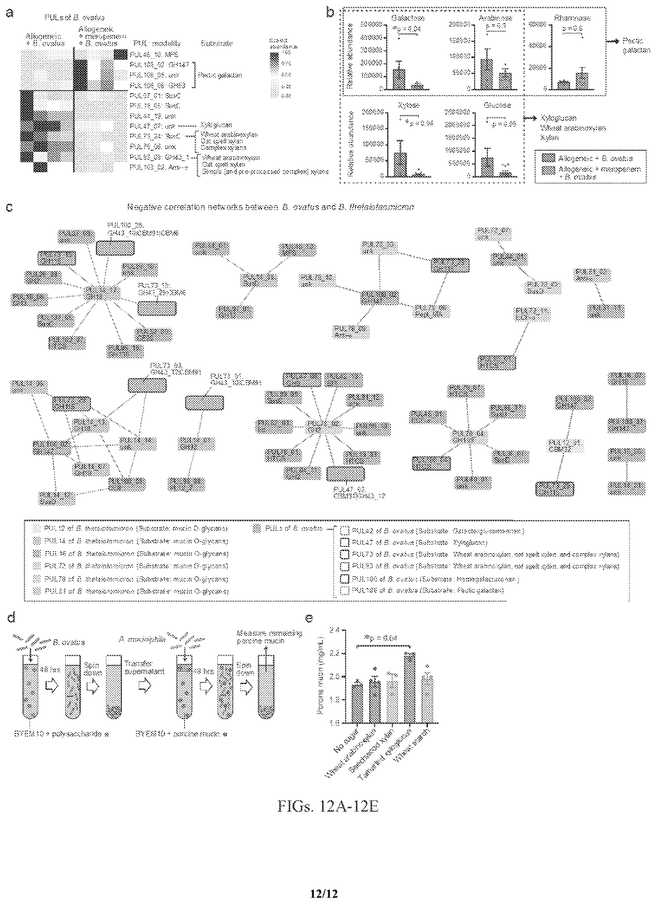

Interestingly, in contrast to B. theta, B. ovatus is known to have the ability to degrade xylose-comprising polysaccharides^{17,29} and the inventors previously found that supplementation of xylose ameliorates GVHD severity in meropenem-treated allogeneic mice by suppressing mucus-degrading functionalities in B. theta¹⁶. This led us to hypothesize that the ability of B. ovatus to degrade xylose-comprising polysaccharides could ameliorate GVHD via production of xylose in the colonic lumen. The inventors quantified gene expression of B. ovatus using microbial RNA sequencing of stool samples and found that B. ovatus in meropenem-untreated mice showed higher expression of PULs that perform degradation of xylose-comprising polysaccharides (FIG. 6A), presumably reflecting enriched xylose-comprising polysaccharides in the intestinal environment of meropenem-untreated mice compared to meropenem-treated mice (FIGS. 5C, 11C). These data suggested that expression of B. ovatus PULs was altered depending on intestinal levels of carbohydrates, which are possibly modulated by nutritional intake and metabolism by other bacteria. Despite lower expression of B. ovatus PULs that degrade xylose-comprising polysaccharides in meropenem-treated mice compared to meropenem-untreated mice, meropenem-treated allo-HSCT mice sufficiently showed reduced abundances of B. theta probably either due to still sufficient functionalities to degrade xylose-comprising polysaccharides or due to higher abundances of B. ovatus in meropenem-treated mice. To investigate potential interactions between B. ovatus and B. theta, the inventors performed network analysis between expression of B. ovatus PULs and B. theta PULs that were related to the degradation of mucin O-glycans. Given the hypothesis that B. ovatus was performing metabolic functions that inhibited utilization of mucins by B. theta, the inventors were particularly interested in PULs of B. ovatus that were negatively associated with PULs of B. theta that participate in degradation of mucin O-glycans. The inventors found that multiple genes belonging to PULs of B. ovatus involved in metabolising xylose-comprising polysaccharides, including xyloglucan, wheat arabinoxylan, oat spelt xylan, and complex xylans, were negatively correlated with genes belonging to PULs of B. theta involved in degradation of mucin O-glycans^{29,30} (FIG. 12C). These B. ovatus genes encoded enzymes such as beta-glucosidase, beta-galactosidase, and beta-xylosidase (Table 5). This led us to ask if degradation of xylose-comprising polysaccharides by B. ovatus could produce metabolic byproducts that suppress mucin glycan utilization by B. theta in vitro. The inventors evaluated the effects of combining minimal media supplemented with porcine gastric mucin with media conditioned by B. ovatus for 48 hours in the presence of wheat arabinoxylan, beechwood xylan or tamarind xyloglucan, which are composed of xylose, or wheat starch, which is not composed of xylose (FIG. 6A). Interestingly, culture media supplemented with wheat arabinoxylan or tamarind xyloglucan followed by B. ovatus-conditioning each significantly suppressed mucin degradation by B. theta, while culture medium supplemented with beechwood xylan and wheat starch followed by B. ovatus-conditioning did not suppress mucin degradation by B. theta (FIG. 6B). These contrasts were correlated with differences in concentrations of xylose in culture media after conditioning by B. ovatus (FIG. 6C). Interestingly, mucin degradation by A. muciniphila was suppressed only by culture medium supplemented with tamarind xyloglucan after B. ovatus-conditioning, suggesting that suppression of mucus-degrading functionalities in A. muciniphila is independent of production of xylose (FIGS. 12D, 12E).

TABLE 5

| network | B. theta PUL | B. ovatus PUL |
|---|---|---|
| 1 | PUL12_01_CBM32: putative | PUL73_29_GH115: xylan a-1,2-(4-O- |
| | chitobiase | methyl)-glucuronidase |
| | | PUL108_02_GH147: Cellulase |
| | | (glycosyl hydrolase family 5) |
| 2 | PUL14_01_GH92: a- | PUL96_08_PL12_2: Heparin-sulfate |
| | mannosidase/hypothetical protein | lyase precursor |
| | | PUL108_02_GH147: b- |
| | | galactosidase/Cellulase (glycosyl |
| | | hydrolase family 5) |
| 3 | PUL14_06_unk: hypothetical | PUL73_03_GH43_12\|CBM91: a-L- |
| | protein | arabinofuranosidase/Beta-xylosidase |
| | PUL14_07_GH18: hypothetical | PUL73_29_GH115: xylan a-1,2-(4-O- |
| | protein | methyl)-glucuronidase |
| | PUL14_12_SusD: hypothetical | PUL108_02_GH147: b- |
| | protein | galactosidase/Cellulase (glycosyl |
| | | hydrolase family 5) |
| | PUL14_13_GH18: mannosyl- | PUL100_08_CE8: Pectinesterase A |
| | glycoprotein-endo-b-N- | precursor |
| | acetylglucosaminidase/hypothetical | |
| | protein | |
| | PUL14_14_unk: hypothetical | |
| | protein | |
| 4 | PUL14_08_SusD: hypothetical | PUL44_01_unk: ribulose-1,5- |
| | protein | biphosphate synthetase |
| | | PUL46_10_MFS: Inner membrane |
| | | symporter YicJ |
| | | PUL97_01_GH32: Levanase precursor |
| 5 | PUL14_17_GH18: putative | PUL18_06_GH3: b- |
| | secreted endoglycosidase | glucosidase/Thermostable beta- |
| | | glucosidase B |
| | | PUL26_08_GH2: Beta-galactosidase |
| | | PUL27_06_unk: Bacterial Ig-like |
| | | domain (group 2) |
| | | PUL52_09: CE20: hypothetical protein |
| | | PUL73_16_GH115: hypothetical |
| | | protein |
| | | PUL73_18: GH43_29\|CBM6: |
| | | Arabinoxylan arabinofuranohydrolase |
| | | precursor |
| | | PUL85_19_GH130: Beta-1,4- |
| | | mannooligosaccharide phosphorylase |
| | | PUL97_10_unk: Fructosamine kinase |
| | | FrlD |
| | | PUL100_25_GH43_10\|CBM91\|CBM6: |
| | | Beta-xylosidase |
| | | PUL102_07_HTCS: Sensor histidine |
| | | kinase TmoS |
| | | PUL107_05_SusC: —————— |
| | | TonB dependent receptor |
| 6 | PUL16_06_unk: hypothetical | PUL44_01_unk: CoA binding domain |
| | protein | protein |
| | PUL16_07_GH18: putative endo- | PUL108_02_GH147: Cellulase |
| | beta-N-acetylglucosaminidase F1 | (glycosyl hydrolase family 5) |
| | precursor (mannosyl-glycoprotein | |
| | endo-beta-N-acetyl- | |
| | glucosaminidase F1) | |
| 7 | PUL72_02_SusD: hypothetical | PUL44_01_unk: CoA binding domain |
| | protein | protein |
| | PUL72_07_unk: hypothetical | |
| | protein | |
| 8 | PUL72_11_ECF-s: RNA | PUL93_01_HTCS: Sensor histidine |
| | polymerase ECF-type sigma factor | kinase TmoS |
| 9 | PUL78_05_Pept_MA: hypothetical | PUL73_29_GH115: xylan a-1,2-(4-O- |
| | protein | methyl)-glucuronidase |
| | PUL78_06_unk: hypothetical | PUL108_02_GH147: b- |
| | protein | galactosidase/Cellulase (glycosyl |
| | | hydrolase family 5) |
| | PUL78_09_Anti-s: putative anti- | |
| | sigma factor | |
| | PUL78_10_unk: hypothetical | |
| | protein | |
| 10 | PUL78_02_GH2: beta- | PUL04_11_GH2: Beta-galactosidase |
| | galactosidase | PUL19_03_HTCS: Sensor histidine |
| | | kinase TmoS |
| | | PUL42_10_EPI: Cellobiose 2- |
| | | epimerase |
| | | PUL47_03_CBM91\|GH43_12: a-L- |
| | | arabinofuranosidase/Beta-xylosidase |
| | | PUL47_08_GH9: xyloglucan-specific |
| | | endo-b-1,4-glucanase/endo- |
| | | xyloglucanase/Endoglucanase D |
| | | precursor |
| | | PUL62_03_Int: Tyrosine recombinase |
| | | XerD |
| | | PUL78_01_HTCS: Sensor histidine |
| | | kinase TodS |
| | | PUL91_12_unk: Miniconductance |
| | | mechanosensitive channel YbdG |
| | | PUL99_09_SusC: TonB dependent |
| | | receptor |
| | | PUL99_10_unk: KilA-N domain |
| | | protein |
| 11 | PUL78_04_GH109: putative | PUL36_07_SusD: SusD family protein |
| | oxidoreductase (putative secreted | PUL40_01_unk: HTH-type |
| | protein | transcriptional regulator DegA |
| | | PUL45_01_ECF-s: RNA polymerase |
| | | sigma factor |
| | | PUL79_07_HTCS: Sensor histidine |
| | | kinase TodS |
| | | PUL96_07_SusD: SusD family protein |
| | | PUL100_04_HTCS: Sensor histidine |
| | | kinase TmoS |
| 12 | PUL81_02_Anti-s: putative anti- | PUL31_13_unk: 1,5-anhydro-D- |
| | sigma factor | fructose reductase |

The inventors then asked what direct effects B. ovatus had in vivo on modulating gene expression in B. theta. The inventors turned to gnotobiotic mice and evaluated fecal RNA transcripts in germ-free mice 2 weeks after introducing either B. theta alone or B. ovatus as well as B. theta (FIG. 6D). The inventors found that introduction of B. ovatus resulted in B. theta significantly downregulating PULs involved in degradation of mucin O-glycans (FIG. 6E). Furthermore, to investigate whether the capability to degrade xylose-comprising polysaccharides of B. ovatus was necessary to mitigate GVHD severity, the inventors generated a xylan-PUL-deficient strain of B. ovatus²⁹. GF mice that were administered xylan-PUL-deficient B. ovatus showed significantly reduced levels of xylose in the colonic lumen (FIGS. 6F, 6G). The inventors then administered xylan-PUL-deficient B. ovatus to meropenem-treated allogeneic mice and evaluated survival (FIG. 6H). Interestingly, xylan-PUL-deficient B. ovatus failed to improve survival in meropenem-treated mice, in contrast to control wild-type B. ovatus (ATCC8483 with genetic deletion of thymidine kinase, FIG. 6I). Altogether, these data suggested that after introduction to meropenem-treated allo-HSCT mice, B. ovatus produces a carbohydrate-enriched intestinal environment in the colonic lumen by degrading dietary-derived polysaccharides such as xylose-comprising polysaccharides, leading to inhibition of mucin utilization by mucus-degrading bacteria such as B. theta and A. muciniphila, ultimately resulting in amelioration of disrupted microbiota-induced severe GVHD.

Finally, to genetically assess whether aGI-GVHD patient-derived B. ovatus strains would be predicted to be capable of degrading xylose-comprising polysaccharides, similar to MDA-HVS B0001 and the ATCC strain of B. ovatus (ATCC 8483), the inventors investigated B. ovatus MAGs from 3 responder patients. Importantly, the inventors found that MAGs from each patient included genes involved in degradation of xylose-comprising polysaccharides, indicating that each patient harbored B. ovatus strains with the potential to mitigate aGI-GVHD through their capability to degrade xylose-comprising polysaccharides.

### Example 6

### Genus Bacteroides Enriched Microbiome is Associated with Favorable Treatment Response of aGI-GVHD in Allo-HSCT Patients

Allo-HSCT is a curative therapy for high-risk hematological malignancies, but complications such as infections and GVHD continue to limit its success. The intestinal microbiota is an important modulator of GVHD, and broad-spectrum antibiotics are known to increase the incidence of aGI-GVHD by compromising several functions of an intact intestinal microbiota, resulting in alterations to the intestinal environment including reduced concentrations of metabolic products in the colonic lumen¹⁶. Indeed, the colonic luminal levels of short-chain fatty acids (SCFAs), especially butyrate and propionate, have been shown to suppress colonic GVHD^{31,32}. The poor prognosis of severe aGI-GVHD underlines the need to better understand how intestinal microbes can help suppress GVHD in allo-HSCT.

In this study, the inventors investigated the impact of the intestinal microbiota on treatment responsiveness of aGI-GVHD using clinical microbiome data. In the analysis of prospectively collected fecal samples from a cohort of aGI-GVHD patients, the inventors found that an altered microbiome profile at presentation of aGI-GVHD with lower microbial diversity and disrupted composition, accompanied by prior treatment with antibiotics, was significantly associated with developing steroid-refractory GVHD, whereas a higher abundance of the commensal species B. ovatus, commonly found in normal individuals, was significantly associated with improved GVHD response to steroid therapy. Consistent with this result, B. ovatus has previously been associated with a reduced incidence of GVHD³³. However, it has not been well-studied whether B. ovatus can mechanistically suppress severe GVHD.

Some prior studies have reported that B. ovatus can mediate multiple beneficial functions in maintaining intestinal homeostasis in the host via production of indole-3-acetic acid or sphingolipid production^{28,34}. Here, in a murine model, the inventors found that introduction of B. ovatus resulted in improved survival in meropenem-treated allo-HSCT mice but not in meropenem-untreated allo-HSCT mice. This suggested that B. ovatus helped suppress GVHD only in hosts with a disrupted microbiota, and that a key function of B. ovatus may be related to mechanisms underlying aggravated colonic GVHD in the setting of antibiotic injury. Unlike B. ovatus, B. theta is known to be capable of utilizing host-derived glycans^{35,36}, and was found to aggravate colonic GVHD in the prior study¹⁶. In this study, the inventors found that in the setting of an antibiotic-disrupted microbiota with expansion of mucus-degrading B. theta, the introduction of B. ovatus ameliorated the severity of colonic GVHD via polysaccharide degradation, thus producing abundant monosaccharides and improving the intestinal metabolomic environment in allo-HSCT.

In order to evaluate the potential for causality, the inventors utilized a murine GVHD model combined with in vitro assays and were able to confirm that B. ovatus ameliorated meropenem-aggravated colonic GVHD via xylose-comprising polysaccharide degradation. However, B. ovatus has a broad ability to play a role in not only carbohydrate degradation but also functions in generation of tryptophan metabolites²⁸, sphingolipids³⁴, SCFAs³⁷, and bile salt hydrolase³⁸ and can impact secretion of fecal immunoglobulin A³⁹. In addition, although the inventors have found that B. ovatus can ameliorate GVHD aggravated by a dysbiotic microbiota in a murine model, it remains to be seen whether introduction of B. ovatus can impact on steroid therapy response for aGI-GVHD. Further studies will be needed to fully understand the influence of the intestinal microbiota with regard to response to therapy.

In summary, an antibiotic-disrupted microbiota caused by carbapenems including meropenem increased the severity of intestinal GVHD and was associated with treatment-refractory aGI-GVHD in patients. Mouse modeling demonstrated that introducing B. ovatus can ameliorate the severity of GVHD in a model of meropenem-aggravated colonic GVHD. This understanding of how specific bacteria such as B. ovatus can reduce intestinal inflammation should facilitate the development of new strategies to better prevent and treat this important limitation of allo-HSCT.

### Example 6

### Patient Information

TABLE 1
Patient characteristics of all allo-HSCT patients with aGI-GVHD.

| | n = 37 | |
|---|---|---|
| Median age (range), y | 55 | (22-74) |
| Male, n (%) | 26 | (70%) |
| Donor type, n (%) | | |
| MRD | 11 | (30%) |
| MUD | 20 | (54%) |
| Haplo | 6 | (16%) |
| Cell source, n (%) | | |
| Bone marrow | 4 | (11%) |
| Peripheral blood | 33 | (89%) |
| Conditioning, n (%) | | |
| Myeloablative | 24 | (65%) |
| Non-myeloablative | 13 | (35%) |
| GVHD prophylaxis, n (%) | | |
| PTCy/Tacrolimus | 8 | (22%) |
| PTCy/Tacrolimus/MMF | 8 | (22%) |
| Tacrolimus/MTX | 9 | (24%) |
| Tacrolimus/MTX/ATG | 5 | (14%) |
| Tacrolimus/MMF | 5 | (14%) |
| Tacrolimus/MMF/ATG | 2 | (5%) |
| Median day of aGI-GVHD | 36 | (13-367) |
| onset (range) | | |
| aGI-GVHD clinical | | |
| stages, n (%) | | |
| Stage 0-2 | 25 | (68%) |
| Stage 3-4 | 11 | (30%) |
| Unknown | 1 | (3%) |
| Histology grades of the | | |
| colon, n (%) | | |
| Grade 0-2 | 28 | (76%) |
| Grade 3-4 | 9 | (24%) |

ATG, anti-thymocyte globulin; GVHD, graft-versus-host disease; aGI-GVHD, acute gastrointestinal GVHD; Haplo, human leukocyte antigen (HLA)-haploidentical related donor; MRD, HLA-matched related donor; MTX, methotrexate; MMF, mycophenolate mofetil; MUD, HLA-matched unrelated donor; PTCy, post-transplant cyclophosphamide.

TABLE 2
Patient characteristics of allo-HSCT patients who
were classified into clusters 1 and 2 by intestinal
microbiome profiling at the onset of aGI-GVHD.

| | Cluster 1 | | Cluster 2 | | P |
|---|---|---|---|---|---|
| | (n = 9) | | (n = 28) | | value |
| Median age (range), y | 60 | (46-69) | 51 | (22-74) | 0.05 |
| Male, n (%) | 7 | (78%) | 19 | (66%) | 0.01 |
| Donor type, n (%) | | | | | 0.9 |
| MRD | 3 | (33%) | 8 | (28%) | |
| MUD | 5 | (56%) | 15 | (54%) | |
| Haplo | 1 | (11%) | 5 | (18%) | |
| Cell source, n (%) | | | | | 1.0 |
| Bone marrow | 1 | (11%) | 3 | (11%) | |
| Peripheral blood | 8 | (89%) | 25 | (89%) | |
| Conditioning, n (%) | | | | | 1.0 |
| Myeloablative | 6 | (66%) | 18 | (64%) | |
| Non-myeloablative | 3 | (33%) | 10 | (36%) | |
| GVHD prophylaxis, n (%) | | | | | 0.9 |
| PTCy/Tacrolimus | 2 | (22%) | 6 | (21%) | |
| PTCy/Tacrolimus/MMF | 3 | (33%) | 5 | (18%) | |
| Tacrolimus/MTX | 3 | (33%) | 6 | (21%) | |
| Tacrolimus/MTX/ATG | 0 | (0%) | 5 | (18%) | |
| Tacrolimus/MMF | 0 | (0%) | 5 | (18%) | |
| Tacrolimus/MMF/ATG | 1 | (11%) | 1 | (3%) | |
| Median day of aGI-GVHD | 27 | (14-127) | 41 | (13-367) | 0.04 |
| onset (range) | | | | | |
| aGI-GVHD clinical | | | | | 0.8 |
| stages, n (%) | | | | | |
| Stage 0-2 | 7 | (77%) | 18 | (64%) | |
| Stage 3-4 | 2 | (22%) | 9 | (32%) | |
| Unknown | 0 | (0%) | 1 | (4%) | |
| Histology grades of | | | | | 0.4 |
| the colon, n (%) | | | | | |
| Grade 0-2 | 8 | (88%) | 20 | (71%) | |
| Grade 3-4 | 1 | (11%) | 8 | (29%) | |

Non-repeated ANOVA was used to compare continuous variables, while chi-square or Fisher exact test was used to analyze the frequency distribution between categorical variables. P-value under 0.05 was considered statistically significant. ATG, anti-thymocyte globulin; GVHD, graft-versus-host disease; aGI-GVHD, acute gastrointestinal GVHD; Haplo, human leukocyte antigen (HLA)-haploidentical related donor; MRD, HLA-matched related donor; MTX, methotrexate; MMF, mycophenolate mofetil; MUD, HLA-matched unrelated donor; PTCy, post-transplant cyclophosphamide.

TABLE 3
Patient characteristics of allo-HSCT patients who underwent
intestinal microbiome profiling at the onset of aGI-GVHD.

| | Steroid- | | Steroid- | | |
|---|---|---|---|---|---|
| | responsive | | refractory | | P |
| | (n = 20) | | (n = 17) | | value |
| Median age (range), y | 62 | (46-74) | 45 | (22-71) | 0.0001 |
| Male, n (%) | 11 | (55%) | 13 | (77%) | 0.3 |
| Donor type, n (%) | | | | | 0.6 |
| MRD | 5 | (25%) | 6 | (35%) | |
| MUD | 13 | (65%) | 7 | (41%) | |
| Haplo | 2 | (10%) | 4 | (24%) | |
| Cell source, n (%) | | | | | 1.0 |
| Bone marrow | 2 | (10%) | 2 | (10%) | |
| Peripheral blood | 18 | (86%) | 17 | (85%) | |
| Conditioning, n (%) | | | | | 0.7 |
| Myeloablative | 12 | (60%) | 12 | (71%) | |
| Non-myeloablative | 8 | (40%) | 5 | (29%) | |
| GVHD prophylaxis, n (%) | | | | | 0.5 |
| PTCy/Tacrolimus | 7 | (35%) | 1 | (6%) | |
| PTCy/Tacrolimus/MMF | 4 | (20%) | 4 | (24%) | |
| Tacrolimus/MTX | 4 | (20%) | 5 | (29%) | |
| Tacrolimus/MTX/ATG | 1 | (5%) | 4 | (24%) | |
| Tacrolimus/MMF | 2 | (10%) | 3 | (18%) | |
| Tacrolimus/MMF/ATG | 2 | (10%) | 0 | (0%) | |
| Median day of aGI-GVHD | 32 | (14-367) | 42 | (13-253) | 0.13 |
| onset (range) | | | | | |
| aGI-GVHD clinical | | | | | 0.03 |
| stages, n (%) | | | | | |
| Stage 1-2 | 18 | (90%) | 7 | (41%) | |
| Stage 3-4 | 2 | (10%) | 9 | (53%) | |
| Unknown | 0 | (0%) | 1 | (5%) | |
| Histology grades of the | | | | | 0.005 |
| colon, n (%) | | | | | |
| Grade 0-2 | 19 | (95%) | 9 | (53%) | |
| Grade 3-4 | 1 | (5%) | 8 | (47%) | |

Non-repeated ANOVA was used to compare continuous variables, while chi-square or Fisher exact test was used to analyze the frequency distribution between categorical variables. P-value under 0.05 was considered statistically significant. ATG, anti-thymocyte globulin; GVHD, graft-versus-host disease; aGI-GVHD, acute gastrointestinal GVHD; Haplo, human leukocyte antigen (HLA)-haploidentical related donor; MRD, HLA-matched related donor; MTX, methotrexate; MMF, mycophenolate mofetil; MUD, HLA-matched unrelated donor; PTCy, post-transplant cyclophosphamide.

TABLE 4
Patient characteristics of allo-HSCT patients who underwent
intestinal microbiome profiling at the onset of GI-GVHD
at Memorial Sloan Kettering Cancer Center.

| | Steroid- | | Steroid- | | |
|---|---|---|---|---|---|
| | responsive | | refractory | | P |
| | (n = 26) | | (n = 6) | | value |
| Median age (range), y | 56 | (21-78) | 46 | (25-65) | 0.1 |
| Male, n (%) | 16 | (62%) | 4 | (67%) | >0.9 |
| Donor type, n (%) | | | | | 0.8 |
| MRD | 3 | (12%) | 0 | (0%) | |
| MUD | 9 | (35%) | 2 | (33%) | |
| MMUD | 5 | (19%) | 0 | (0%) | |
| Cord | 9 | (35%) | 4 | (67%) | |
| Cell source, n (%) | | | | | 0.2 |
| Bone marrow | 0 | (0%) | 1 | (17%) | |
| Peripheral blood | 17 | (65%) | 1 | (17%) | |
| Cord blood* | 9 | (35%) | 4 | (67%) | |
| Conditioning, n (%) | | | | | 0.7 |
| Ablative | 1 | (4%) | 0 | (0%) | |
| Non-ablative | 2 | (8%) | 0 | (0%) | |
| Reduced intensity | 23 | (89%) | 6 | (100%) | |
| GVHD prophylaxis, n (%) | | | | | 0.9 |
| Cyclosporine/MMF | 9 | (35%) | 4 | (67%) | |
| PTCy/Sirolimus/MMF | 1 | (4%) | 0 | (0%) | |
| PTCy/Tacrolimus/MMF | 2 | (8%) | 0 | (0%) | |
| Tacrolimus/MTX | 6 | (23%) | 2 | (33%) | |
| Tacrolimus/MTX/MMF | 3 | (12%) | 0 | (0%) | |
| Tacrolimus/Sirolimus/MTX** | 5 | (19%) | 0 | (0%) | |
| GI-GVHD clinical | | | | | 0.001 |
| stages, n (%) | | | | | |
| Stage 1-2 | 23 | (88%) | 1 | (17%) | |
| Stage 3-4 | 3 | (12%) | 5 | (83%) | |
| First-line therapy for | | | | | 0.9 |
| GI-GVHD, n (%) | | | | | |
| Methylprednisolone | 11 | (42%) | 3 | (50%) | |
| Prednisone | 1 | (4%) | 1 | (17%) | |
| Budesonide | 14 | (54%) | 2 | (33%) | |

*Cord blood includes PBSC CD34⁺ related haploidentical/Double cord blood graft (n = 3)
**1 patient additionally received MMF for GVHD prophylaxis.
Non-repeated ANOVA was used to compare continuous variables, while chi-square or Fisher exact test was used to analyze the frequency distribution between categorical variables. P-value under 0.05 was considered statistically significant. GVHD, graft-versus-host disease; GI-GVHD, gastrointestinal GVHD; MRD, HLA-matched related donor; MTX, methotrexate; MMF, mycophenolate mofetil; MUD, HLA-matched unrelated donor; PTCy, post-transplant cyclophosphamide.

### Example 7

### Materials and Methods for Certain Aspects

### Retrospective Study Design

A total of 37 aGI-GVHD patients who underwent allo-HSCT during 2017 to 2019 at MD Anderson Cancer Center provided stool samples for the biorepository, and these patient stool samples were analyzed retrospectively as a discovery cohort. Acute GVHD was diagnosed by clinical and/or pathological findings and graded according to standard criteria⁴⁰. These patients included 28 with classic aGI-GVHD and 9 with late-onset aGI-GVHD by National Institutes of Health consensus criteria¹⁸. The inventors classified patients by steroid responsiveness to GVHD, including 20 patients who were steroid-responsive and 17 patients who were steroid-refractory. The inventors determined treatment response as previously reported¹⁹: briefly, a lack of response on the basis of organ assessment after at least 3 days of high-dose systemic glucocorticoid therapy; a lack of improvement after 7 days; or treatment failure during steroid tapering or an inability to taper the dose to <0.5 mg/kg/day of methylprednisolone. All patients received initial therapy with methylprednisolone or prednisone at 2 mg/kg/day followed by tapering per institutional guidelines. As another cohort, a total of 16 aGI-GVHD patients who underwent allo-HSCT during 2017 to 2020 at MD Anderson Cancer Center provided stool samples collected on day 14 after allo-HSCT for the biorepository, and these patient stool samples were analyzed retrospectively. Signed informed consent was provided by all study participants including healthy volunteers, and this study was approved by The University of Texas MD Anderson's Institutional Review Board. As a validation cohort, the publicly available whole genome sequencing data of 32 fecal samples collected at the onset of aGI-GVHD at Memorial Sloan Kettering Cancer Center were investigated²².

### Human Samples

Samples were collected from patients undergoing allo-HSCT and healthy volunteers and stored at 4° C. for 24-48 hours until aliquoted for long-term storage at −80° C.

### Mice

Female C57BL/6J (B6: H-2^{b}) and B6D2F1 (H-2^{b/d}, CD45.2⁺) were purchased from The Jackson Laboratory (Bar Harbor, ME). Mice were group housed and provided with standard chow (LabDiet 5053) and water. Six- to 12-week-old female C57BL/6 germ-free mice for murine studies were provided by the gnotobiotic facility of Baylor College of Medicine (Houston, TX). Gnotobiotic mice were provided with autoclaved standard chow (LabDiet 5V0F) and water. All animal experiments were performed under the Guide for the Care and Use of Laboratory Animals Published by the National Institutes of Health and was approved by the Institutional Animal Care and Use Committee. Experiments in this manuscript were performed in a non-blinded fashion.

### Antibiotics Administration

Meropenem was dissolved with phosphate buffer, pH 8.0, and given at a concentration of 0.625 g/L in drinking water from day 3 to day 15 after transplant.

### HSCT

Mice received transplants as previously described⁴¹. In brief, after receiving myeloablative total-body irradiation (11 Gray) delivered in 2 doses at 4-hour intervals, B6D2F1 (H-2^{b/d}) mice were intravenously injected with 5×10⁶ bone marrow cells and 5×10⁶ splenocytes from allogeneic B6 (H-2^{b}) donors. Female mice that were 8 to 12 weeks old were allocated randomly to each experimental group, ensuring the mean body weight in each group was similar. Total body radiotherapy was performed using a Shepherd Mark I, Model 30, ¹³⁷Cs irradiator. Mice were maintained in specific pathogen-free (SPF) conditions and received normal chow (LabDiet PicoLab Rodent Diet 20 5053, Lab Supply). Survival after HSCT was monitored daily, and the degree of clinical GVHD was assessed weekly using an established scoring system⁴².

### Histological and Immunohistochemistry Analysis

For evaluation of mucus thickness, colonic sections containing stool pellets were fixed in methanol-Carnoy fixative composed of methanol (60%), chloroform (30%) and glacial acetic acid (10%) and 5 μm sections were made and stained with periodic acid-Schiff (PAS). Sections were imaged using an Aperio AT2. Mucus thickness of the colonic sections was measured using eSlide Manager Version 12.4.3.5008. Eight measurements per image were taken and averaged over the entire usable colon surface. For pathological analysis, samples of the colon were fixed in 10% formalin, embedded in paraffin, sectioned, and stained with hematoxylin and eosin (H&E). Pathology scores were quantified by a blinded pathologist.

Sequencing of 16S rRNA Gene Amplicons

Fecal samples that were collected from patients and mice were weighed before DNA isolation. In brief, genomic DNA was isolated using the QIAamp DNA mini kit (51306, Qiagen) according to the manufacturer's protocol, which was modified to include an intensive bead-beating lysis step. The V4 region of the 16S rRNA gene was amplified by PCR from 100 ng of extracted genomic DNA using 515 forward and 806 reverse primer pairs 43. The quality and quantity of the barcoded amplicons were assessed on an Agilent 4200 TapeStation system and Qubit Fluorometer (Thermo Fisher Scientific), and libraries were prepared after pooling at equimolar ratios. The final libraries were purified using QIAquick gel extraction kit (28706X4, Qiagen) and sequenced with a 2×250 base pair paired-end protocol on the Illumina MiSeq platform.

### Microbiome Data Analysis

Sequencing data from paired-end reads were de-multiplexed using QIIME 2⁴⁴. Merging of paired-end reads, dereplicating, and length filtering was performed using VSEARCH 2.17.1⁴⁵. Following de-noising and chimera calling using the unoise3 command⁴⁶ unique sequences were taxonomically classified with mothur⁴⁷ using the Silva database⁴⁸ version 138. Weighted UniFrac distances⁴⁹ were determined using QIIME 2, visualized using PCoA, and evaluated for statistical significance using PERMANOVA testing. For differential abundance analysis, abundances of sequences belonging to taxonomical groups were included for analysis using DESeq2 and adjusted for multiple comparisons using the method of Benjamini and Hochberg. Patient microbiome data were classified into 2 clusters using the hcluster function by the amap library of R.

### Quantification of Fecal Bacterial Density

Genomic DNA was isolated from stool as described above. qPCR was performed as previously described⁵⁰. In brief, 16S rRNA gene sequences were amplified from total fecal DNA using the primers 926F (5′-AAACTCAAAKGAATTGACGG-3′) and 1062R (5′-CTCACRRCACGAGCTGAC-3′). Real-time PCR was carried out in 96-well optical plates on QuantStudio Flex 6 RT-PCR (Thermo Fisher) and KAPA SYBR FAST Master Mix (Roche). The PCR conditions included one initial denaturing step of 10 min at 95° C. and 40 cycles of 95° C. for 20 sec and 60° C. for 1 min. Melting-curve analysis was performed after amplification. To determine bacterial density, a plasmid with a 16S rRNA gene of a murine Blautia isolate was generated in the pCR4 backbone and used as a standard.

### Culturing of Bacteria

Bacteroides ovatus (MDA-HVS B0001) was isolated and cultured from healthy volunteer's stool samples in a Whitley anaerobic chamber (10% H₂, 5% Cl₂ and 85% N₂). Human-derived B. ovatus (ATCC 8483) and human-derived B. theta (ATCC 29148) were purchased from American Type Culture Collection (ATCC) and xylan-PUL deficient B. ovatus and wild-type B. ovatus (ATCC8483 with gene deletion of thymidine kinase) were provided from Dr. Eric Martens (University of Michigan Medical School, Ann Arbor, Michigan). Mouse-derived B. theta (MDA-JAX BT001) and mouse-derived A. muciniphila (MDA-JAX AM001) were previously isolated¹⁶. Bacterial number was quantified using a Nexcelom Cellometer cell counter with SYTO BC dye and propidium iodide. Bacterial growth experiments were performed in a liquid media, BYEM10, composed of a hybrid of BHI and M10 supplemented with yeast extract as previously described^{16,51}. Bacteria were cultured up to 24 or 48 hours at a starting concentration of 1×10⁶ bacteria/ml in BYEM10 broth (pH 7.2) with or without 5 mg/ml of porcine gastric mucin (M1778, Sigma-Aldrich), wheat arabionoxylan (wheat flour; low viscosity; Megazyme), xylan (Beechwood; Megazyme), xyloglucan (Tamarind; Megazyme), or starch (wheat; Sigma-Aldrich). Optical densities (OD_{600 nm}) of bacterial cultures were measured with a BioTek Epoch 2 plate reader.

### Mucin Degradation Assay

Levels of mucin glycans in culture supernatants were determined by a PAS-based colorimetric assay as previously described^{16,51}. Briefly, culture supernatants were centrifuged at 20,000 g for 10 minutes at 4° C. and collected. To perform mucin precipitation, 500 μl of culture supernatants was mixed with 1 ml of molecular grade ethanol and incubated at −30° C. for overnight. Culture supernatants were centrifuged at 20,000 g for 10 minutes at 4° C. Mucin-containing pellets were washed with 1 ml of molecular grade ethanol twice and resuspended in 500 μl of PBS. A total of 10 μl of washed culture supernatants was transferred into a round-bottom 96-well plate containing 15 μl of PBS. Serially diluted porcine gastric mucin (Sigma-Aldrich) standards were prepared. Freshly prepared 0.06% periodic acid in 7% acetic acid was added and incubated at 37° C. for 90 min, followed by 100 μl of Schiff's reagent (84655, Sigma-Aldrich) and incubation at room temperature for 40 min. Absorbance was measured at 550 nm using a BioTek Synergy HTX plate reader.

### Analysis of Carbohydrates by IC-MS

To determine the relative abundance of carbohydrates in mouse fecal samples, extracts were prepared and analyzed by ultrahigh-resolution mass spectrometry. Fecal pellets were homogenized with a Precellys Tissue Homogenizer. Metabolites were extracted using 1 ml of ice-cold 80/20 (v/v) methanol/water. Extracts were centrifuged at 17,000 g for 5 min at 4° C., and supernatants were transferred to clean tubes, followed by evaporation to dryness under nitrogen. Dried extracts were reconstituted in deionized water, and 5 μl was injected for analysis by IC-MS. IC mobile phase A (MPA; weak) was water, and mobile phase B (MPB; strong) was water containing 100 mM KOH. A Thermo Scientific Dionex ICS-5000+ system included a Thermo CarboPac PA20-Fast column (4 μm particle size, 100×2 mm) with the column compartment kept at 30° C. The autosampler tray was chilled to 4° C. The mobile phase flow rate was 200 μl/min, and the gradient elution program was: 0-0.5 min, 1% MPB; 0.5-10 min, 1%-5% MPB; 10-15 min, 5%-95% MPB; 15-20 min, 95% MPB; 20.5-25, 95-1% MPB. The total run time was 25 min. To assist the desolvation for better sensitivity, methanol was delivered by an external pump and combined with the eluent via a low dead volume mixing tee. Data were acquired using a Thermo Orbitrap Fusion Tribrid Mass Spectrometer under ESI negative ionization mode at a resolution of 240,000. Raw data files were imported to Thermo TraceFinder and Compound Discoverer software for spectrum database analysis. The relative abundance of each metabolite was normalized by sample weight.

### Analysis of Tryptophan Metabolites by LC-HRMS

To determine the relative concentration of tryptophan metabolites in mouse fecal samples, extracts were prepared and analyzed by liquid chromatography coupled with high-resolution mass spectrometry (LC-HRMS). Approximately 50 mg of stool was pulverized on liquid nitrogen, then homogenized with Precellys Tissue Homogenizer. Metabolites were extracted using 0.5 ml of ice-cold 50/50 (v/v) methanol/acetonitrile followed by 0.5 mL 0.1% formic acid in 50/50 (v/v) Acetonitrile/Water. Extracts were centrifuged at 17,000 g for 5 min at 4° C., and supernatants were transferred to clean tubes, followed by evaporation to dryness under nitrogen. Samples were then reconstituted in 50/50 (v/v) methanol/water, then 10 μl was injected into a Thermo Vanquish liquid chromatography (LC) system containing a Waters XSelect HSS T3 2.1×150 mm column with 2.5-μm particle size. MPA was 0.1% formic acid in water. MPB was 100% methanol. The flow rate was 200 μl/min (at 35° C.), and the gradient conditions were: initial 5% MPB, increased to 95% MPB at 15 min, held at 95% MPB for 5 min, and returned to initial conditions and equilibrated for 5 min. The total run time was 25 min. Data were acquired using a Thermo Orbitrap Fusion Tribrid mass spectrometer under ESI positive and negative ionization modes at a resolution of 240,000 with full scan mode. Raw data files were imported into Thermo TraceFinder software for final analysis. The relative concentration of each compound was normalized by stool weight.

### Whole-Genome Sequencing of Patient Fecal Samples

Genomic DNA was isolated from patient fecal samples and purified using a Qiagen Genomic-tip 20/G column, according to the manufacturer's instructions. For short-read Illumina sequencing, libraries were constructed with a Nextera DNA Flex Library Prep Kit (Illumina), according to the manufacturer's protocol. All libraries were quantified with a TapeStation and pooled in equal molar ratios. The final libraries were sequenced with the NovaSeq 6000 platform (Illumina) to produce 2×150 bp paired-end reads, resulting in −5 Gb per sample. In sequencing analysis, sequence reads were filtered by their quality using the VSEARCH 2.17.1. The abundance of taxa, microbial metabolic pathways, and gene expression was profiled by the HUMAnN3. Differential expression profiles were analyzed by the DESeq2 package in R.

Whole-Genome Sequencing of B. ovatus (MDA-HVS B0001)

B. ovatus (MDA-HVS B0001) genomic DNA was isolated and purified using a Qiagen Genomic-tip 20/G column, according to the manufacturer's instructions. For short-read Illumina sequencing, libraries were constructed with a Nextera DNA Flex Library Prep Kit (Illumina, San Diego, CA, USA), according to the manufacturer's protocol. All libraries were quantified with a TapeStation and pooled in equal molar ratios. The final libraries were sequenced with the NovaSeq 6000 platform (Illumina) to produce 2×150 bp paired-end reads, resulting in ˜5 Gb per sample. For long-read Nanopore sequencing, 500 ng of genomic DNA was used for library preparation using the Rapid Sequencing Kit (SQK-RAD004, Oxford Nanopore Technologies). Libraries were loaded into a FLO-MIN106 flow-cell for a 24-h sequencing run on a MinION sequencer platform (Oxford Nanopore Technologies, Oxford, UK). Data acquisition and real-time base calling were carried out by the MinKNOW software version 3.6.5. The fastq files were generated from basecalled sequencing fast5 reads.

Hybrid Assembly and Genome Annotation of B. ovatus (MDA-HVS B0001)

To assemble the complete genome of B. ovatus, Flye version 2.8.2⁵² was used with long reads (Nanopore) and short reads (NovaSeq) combined using default settings. The similarities of the genome of MDA-HVS B0001 to other reference genomes was calculated using blastn for B. (ATCC 8483)⁵³. Open reading frames of B. ovatus (MDA-HVS B0001) were identified using prokka⁵⁴. The genome of B. ovatus and open reading frames were depicted using DNA plotter software⁵⁵.

Metagenome-Assembled Genomes (MAGs) from Patient Stool Datasets

To recover B. ovatus MAGs from patient's stool metagenomic datasets that were previously determined to have a high abundance of B. ovatus, reads were assembled using MEGAHIT⁵⁶ Resulting contigs were mapped to a databases of 494 non-MAG genomes available at GenBank⁵⁷. Contigs mapping >95% to all B. ovatus genomes were retained for further consideration. The matching contigs were mapped to all genomes within Bacteroidales class. Contigs that aligned better to Bacteroidales species other than B. ovatus were removed. A final filter using Kraken2⁵⁸ removed database contaminants. To cluster the B. ovatus genomes, the inventors used MASH to determine the average nucleotide identity (ANI) between all B. ovatus genomes (MAGs+494 entries). Calculated distances were input into Uniform Manifold Approximation and Projection (UMAP)⁵⁹ and a high and low dimensional embedding were calculated. The high dimensional embedding was used by HDBScan⁶⁰ to compute cluster while the low dimensional embedding was used for plotting.

### RNA Sequencing and Analysis

Approximately 30 mg of stool was freshly collected in 700 μl of ice-cold QIAzol containing 200 μl of 0.1-mm-diameter Zirconia Silica beads (11079101z, BioSpec). Samples were bead beaten twice for 2 min with a 30-s interval recovery. Samples were then centrifuged at 12,000 g for 1 min, and the supernatant was collected for RNA isolation using the RNeasy mini kit (74104, Qiagen). RNA was treated on column with DNase I (79254, Qiagen) to eliminate contaminating genomic DNA. RNA quantity and quality were determined using an Agilent 4200 TapeStation system (Agilent). A total of 250 ng of total RNA from mouse stools was used to construct libraries using the Universal Prokaryotic RNA-Seq Library Preparation Kit (9367-32, Tecan) with Unique Dual Indexes (S02480-FG, Tecan), following the manufacturer's protocol. The cDNA libraries were sequenced on the Illumina NovaSeq 6000 system to produce 2×150 bp paired-end reads. Sequence data were demultiplexed using QIIME 2⁴⁴ and their qualities were checked using VSEARCH 2.17.1⁴⁵. Data were filtered and truncated by quality with VSEARCH default settings. The total reads of mouse stool samples were 160896223±93489752 (mean±standard deviation). Sequences of ribosomal RNA were removed using BWA software against prokaryotic ribosomal RNA sequences from prokaryotic RefSeq genomes⁶¹. Sequences of interest were further identified using diamond software version 0.9.24⁶² to align against PULs. Features with percentage identity less than 80% were excluded. The total counts of bacterial isolated samples were 360932±284308 and 966485±617495 in B. ovatus and B. theta, respectively (mean±standard deviation). Aligned mRNA expression changes were calculated using the DESeq2 in R software version 4.1.2 via RStudio version 2022.02.0 Build 443. P values <0.05 were considered statistically significant.

### Network Analysis Using Bacterial RNA Transcripts

The expressions of PULs of B. theta and B. ovatus in meropenem-untreated and -treated mice that received B. ovatus were standardized by relative abundances in each sample. PULs with average expression rates of 1% or less in each group were excluded from the network analysis. Each data set was logit transformed, and then r and p values were calculated by Pearson correlation analysis between B. theta and B. ovatus PULs. P values were corrected by false discovery rate (FDR). PUL combinations showing a corrected p-value of 0.05 or less with a negative r value were depicted using Cytoscape⁶³ for B. theta PULs known to degrade mucin-O-glycan. PUL combinations showing a corrected p-value of 0.05 or less with a negative r value were depicted using Cytoscape {Shannon, 2003, Cytoscape: a software environment for integrated models of biomolecular interaction networks} for B. theta PULs known to degrade mucin-O-glycan.

### Data and Code Availability

16S rRNA sequencing data and RNA sequencing data (PRJNA1000552), whole-genome sequencing data of patient fecal samples (PRJNA973955), and complete genome data (PRJNA1022439) have been deposited at Sequence Read Archive (SRA). All data are publicly available as of the date of publication. Any additional information required to reanalyze the data reported in this paper is available from the lead contact upon request.

### Statistical Analysis

Data were checked for normality and similar variances between groups, and Student t-tests were used when appropriate. Mann-Whitney U tests were used to compare data between two groups when the data did not follow a normal distribution. Kaplan-Meier curves were used to depict survival probabilities, and the log-rank test was applied to compare survival curves. For clinical data analysis, non-repeated ANOVA was used to compare continuous variables, while chi-square or Fisher exact tests were used to analyze the frequency distribution between categorical variables. Analyses were performed using R software version 4.1.2 and Prism version 9.0 (GraphPad Software). P values <0.05 were considered statistically significant.

All of the methods disclosed and claimed herein can be made and executed without undue experimentation in light of the present disclosure. While the compositions and methods of this disclosure have been described in terms of preferred aspects, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1 Holtan, S. G. et al. Disease progression, treatments, hospitalization, and clinical outcomes in acute GVHD: a multicenter chart review. Bone Marrow Transplant 57, 1581-1585 (2022). https://doi.org/10.1038/s41409-022-01764-w
2 Westin, J. R. et al. Steroid-Refractory Acute GVHD: Predictors and Outcomes. Adv Hematol 2011, 601953 (2011). https://doi.org/10.1155/2011/601953
3 MacMillan, M. L. et al. Response of 443 patients to steroids as primary therapy for acute graft-versus-host disease: comparison of grading systems. Biol Blood Marrow Transplant 8, 387-394 (2002). https://doi.org/10.1053/bbmt.2002.v8.pm12171485
4 Jagasia, M. et al. Ruxolitinib for the treatment of steroid-refractory acute GVHD (REACHi): a multicenter, open-label phase 2 trial. Blood 135, 1739-1749 (2020). https://doi.org/10.1182/blood.2020004823
5 Zeiser, R. et al. Ruxolitinib for Glucocorticoid-Refractory Acute Graft-versus-Host Disease. N Engl J Med 382, 1800-1810 (2020). https://doi.org/10.1056/NEJMoa1917635
6 Zheng, D., Liwinski, T. & Elinav, E. Interaction between microbiota and immunity in health and disease. Cell Res 30, 492-506 (2020). https://doi.org/10.1038/s41422-020-0332-7
7 Hooper, L. V., Littman, D. R. & Macpherson, A. J. Interactions between the microbiota and the immune system. Science 336, 1268-1273 (2012). https://doi.org/10.1126/science.1223490
8 Peled, J. U. et al. Microbiota as Predictor of Mortality in Allogeneic Hematopoietic-Cell Transplantation. N Engl J Med 382, 822-834 (2020). https://doi.org/10.1056/NEJMoa1900623
9 Shono, Y. et al. Increased GVHD-related mortality with broad-spectrum antibiotic use after allogeneic hematopoietic stem cell transplantation in human patients and mice. Sci Transl Med 8, 339ra371 (2016). https://doi.org/10.1126/scitranslmed.aaf2311
10 Hidaka, D. et al. The association between the incidence of intestinal graft-vs-host disease and antibiotic use after allogeneic hematopoietic stem cell transplantation. Clin Transplant 32, e13361 (2018). https://doi.org/10.1111/ctr.13361
11 Farowski, F. et al. Impact of choice, timing, sequence and combination of broad-spectrum antibiotics on the outcome of allogeneic haematopoietic stem cell transplantation. Bone Marrow Transplant 53, 52-57 (2018). https://doi.org/10.1038/bmt.2017.203
12 Lee, S. E. et al. Alteration of the Intestinal Microbiota by Broad-Spectrum Antibiotic Use Correlates with the Occurrence of Intestinal Graft-versus-Host Disease. Biol Blood Marrow Transplant 25, 1933-1943 (2019). https://doi.org/10.1016/j.bbmt.2019.06.001
13 Kakihana, K. et al. Fecal microbiota transplantation for patients with steroid-resistant acute graft-versus-host disease of the gut. Blood 128, 2083-2088 (2016). https://doi.org/10.1182/blood-2016-05-717652
14 Spindelboeck, W. et al. Repeated fecal microbiota transplantations attenuate diarrhea and lead to sustained changes in the fecal microbiota in acute, refractory gastrointestinal graft-versus-host-disease. Haematologica 102, e210-e213 (2017). https://doi.org/10.3324/haematol.2016.154351
15 van Lier, Y. F. et al. Donor fecal microbiota transplantation ameliorates intestinal graft-versus-host disease in allogeneic hematopoietic cell transplant recipients. Sci Transl Med 12 (2020). https://doi.org/10.1126/scitranslmed.aaz8926
16 Hayase, E. et al. Mucus-degrading Bacteroides link carbapenems to aggravated graft-versus-host disease. Cell 185, 3705-3719 e3714 (2022). https://doi.org/10.1016/j.cell.2022.09.007
17 Desai, M. S. et al. A Dietary Fiber-Deprived Gut Microbiota Degrades the Colonic Mucus Barrier and Enhances Pathogen Susceptibility. Cell 167, 1339-1353 e1321 (2016). https://doi.org/10.1016/j.cell.2016.10.043
18 Jagasia, M. H. et al. National Institutes of Health Consensus Development Project on Criteria for Clinical Trials in Chronic Graft-versus-Host Disease: I. The 2014 Diagnosis and Staging Working Group report. Biol Blood Marrow Transplant 21, 389-401 e381 (2015). https://doi.org/10.1016/j.bbmt.2014.12.001
19 Harris, A. C. et al. International, Multicenter Standardization of Acute Graft-versus-Host Disease Clinical Data Collection: A Report from the Mount Sinai Acute GVHD International Consortium. Biol Blood Marrow Transplant 22, 4-10 (2016). https://doi.org/10.1016/j.bbmt.2015.09.001
20 Eriguchi, Y. et al. Graft-versus-host disease disrupts intestinal microbial ecology by inhibiting Paneth cell production of alpha-defensins. Blood 120, 223-231 (2012). https://doi.org/10.1182/blood-2011-12-401166
21 Stein-Thoeringer, C. K. et al. Lactose drives Enterococcus expansion to promote graft-versus-host disease. Science 366, 1143-1149 (2019). https://doi.org/10.1126/science.aax3760
22 Burgos da Silva, M. et al. Preservation of the fecal microbiome is associated with reduced severity of graft-versus-host disease. Blood 140, 2385-2397 (2022). https://doi.org/10.1182/blood.2021015352
23 Zocco, M. A., Ainora, M. E., Gasbarrini, G. & Gasbarrini, A. Bacteroides thetaiotaomicron in the gut: molecular aspects of their interaction. Dig Liver Dis 39, 707-712 (2007). https://doi.org/10.1016/j.dld.2007.04.003
24 Pudlo, N. A. et al. Phenotypic and Genomic Diversification in Complex Carbohydrate-Degrading Human Gut Bacteria. mSystems 7, e0094721 (2022). https://doi.org/10.1128/msystems.00947-21
25 Heimesaat, M. M. et al. MyD88/TLR9 mediated immunopathology and gut microbiota dynamics in a novel murine model of intestinal graft-versus-host disease. Gut 59, 1079-1087 (2010). https://doi.org/10.1136/gut.2009.197434
26 Sofi, M. H. et al. A single strain of Bacteroides fragilis protects gut integrity and reduces GVHD. JCI Insight 6 (2021). https://doi.org/10.1172/jci.insight.136841
27 Terrapon, N. et al. PULDB: the expanded database of Polysaccharide Utilization Loci. Nucleic Acids Res 46, D677-D683 (2018). https://doi.org/10.1093/nar/gkx1022
28 Ihekweazu, F. D. et al. Bacteroides ovatus Promotes IL-22 Production and Reduces Trinitrobenzene Sulfonic Acid-Driven Colonic Inflammation. Am J Pathol 191, 704-719 (2021). https://doi.org/10.1016/j.ajpath.2021.01.009
29 Rogowski, A. et al. Glycan complexity dictates microbial resource allocation in the large intestine. Nat Commun 6, 7481 (2015). https://doi.org/10.1038/ncomms8481
30 Martens, E. C. et al. Recognition and degradation of plant cell wall polysaccharides by two human gut symbionts. PLoS Biol 9, e1001221 (2011). https://doi.org/10.1371/journal.pbio.1001221
31 Mathewson, N. D. et al. Gut microbiome-derived metabolites modulate intestinal epithelial cell damage and mitigate graft-versus-host disease. Nat Immunol 17, 505-513 (2016). https://doi.org/10.1038/ni.3400
32 Fujiwara, H. et al. Microbial metabolite sensor GPR43 controls severity of experimental GVHD. Nat Commun 9, 3674 (2018). https://doi.org/10.1038/s41467-018-06048-w
33 Golob, J. L. et al. Stool Microbiota at Neutrophil Recovery Is Predictive for Severe Acute Graft vs Host Disease After Hematopoietic Cell Transplantation. Clin Infect Dis 65, 1984-1991 (2017). https://doi.org/10.1093/cid/cix699
34 Brown, E. M. et al. Bacteroides-Derived Sphingolipids Are Critical for Maintaining Intestinal Homeostasis and Symbiosis. Cell Host Microbe 25, 668-680 e667 (2019). https://doi.org/10.1016/j.chom.2019.04.002
35 Bergstrom, K. S. & Xia, L. Mucin-type O-glycans and their roles in intestinal homeostasis. Glycobiology 23, 1026-1037 (2013). https://doi.org/10.1093/glycob/cwtO45
36 Tailford, L. E., Crost, E. H., Kavanaugh, D. & Juge, N. Mucin glycan foraging in the human gut microbiome. Front Genet 6, 81 (2015). https://doi.org/10.3389/fgene.2015.00081
37 Horvath, T. D. et al. Bacteroides ovatus colonization influences the abundance of intestinal short chain fatty acids and neurotransmitters. iScience 25, 104158 (2022). https://doi.org/10.1016/j.isci.2022.104158
38 Yoon, S. et al. Bile salt hydrolase-mediated inhibitory effect of Bacteroides ovatus on growth of Clostridium difficile. J Microbiol 55, 892-899 (2017). https://doi.org/10.1007/s12275-017-7340-4
39 Yang, C. et al. Fecal IgA Levels Are Determined by Strain-Level Differences in Bacteroides ovatus and Are Modifiable by Gut Microbiota Manipulation. Cell Host Microbe 27, 467-475 e466 (2020). https://doi.org/10.1016/j.chom.2020.01.016
40 Przepiorka, D. et al. 1994 Consensus Conference on Acute GVHD Grading. Bone Marrow Transplant 15, 825-828 (1995).
41 Hayase, E. et al. R-Spondinl expands Paneth cells and prevents dysbiosis induced by graft-versus-host disease. J Exp Med 214, 3507-3518 (2017). https://doi.org/10.1084/jem.20170418
42 Cooke, K. R. et al. An experimental model of idiopathic pneumonia syndrome after bone marrow transplantation: I. The roles of minor H antigens and endotoxin. Blood 88, 3230-3239 (1996).
43 Caporaso, J. G. et al. Ultra-high-throughput microbial community analysis on the Illumina HiSeq and MiSeq platforms. ISME J 6, 1621-1624 (2012). https://doi.org/10.1038/ismej.2012.8
44 Bolyen, E. et al. Reproducible, interactive, scalable and extensible microbiome data science using QIIME 2. Nat Biotechnol 37, 852-857 (2019). https://doi.org/10.1038/s41587-019-0209-9
45 Rognes, T., Flouri, T., Nichols, B., Quince, C. & Mahe, F. VSEARCH: a versatile open source tool for metagenomics. PeerJ 4, e2584 (2016). https://doi.org/10.7717/peerj.2584
46 Edgar, R. C. UNOISE2: improved error-correction for Illumina 16S and ITS amplicon sequencing. bioRxiv, 081257 (2016). https://doi.org/10.1101/081257
47 Schloss, P. D. et al. Introducing mothur: open-source, platform-independent, community-supported software for describing and comparing microbial communities. Appl Environ Microbiol 75, 7537-7541 (2009). https://doi.org/10.1128/AEM.01541-09
48 Quast, C. et al. The SILVA ribosomal RNA gene database project: improved data processing and web-based tools. Nucleic Acids Res 41, D590-596 (2013). https://doi.org/10.1093/nar/gks1219
49 Lozupone, C., Lladser, M. E., Knights, D., Stombaugh, J. & Knight, R. UniFrac: an effective distance metric for microbial community comparison. ISME J 5, 169-172 (2011). https://doi.org/10.1038/ismej.2010.133
50 Yang, Y. W. et al. Use of 16S rRNA Gene-Targeted Group-Specific Primers for Real-Time PCR Analysis of Predominant Bacteria in Mouse Feces. Appl Environ Microbiol 81, 6749-6756 (2015). https://doi.org/10.1128/AEM.01906-15
51 Schwabkey, Z. I. et al. Diet-derived metabolites and mucus link the gut microbiome to fever after cytotoxic cancer treatment. Sci Transl Med 14, eabo3445 (2022). https://doi.org/10.1126/scitranslmed.abo3445
52 Kolmogorov, M., Yuan, J., Lin, Y. & Pevzner, P. A. Assembly of long, error-prone reads using repeat graphs. Nat Biotechnol 37, 540-546 (2019). https://doi.org/10.1038/s41587-019-0072-8
53 Altschul, S. F., Gish, W., Miller, W., Myers, E. W. & Lipman, D. J. Basic local alignment search tool. J Mol Biol 215, 403-410 (1990). https://doi.org/10.1016/S0022-2836(05)80360-2
54 Seemann, T. Prokka: rapid prokaryotic genome annotation. Bioinformatics 30, 2068-2069 (2014). https://doi.org/10.1093/bioinformatics/btul53
55 Carver, T., Thomson, N., Bleasby, A., Berriman, M. & Parkhill, J. DNAPlotter: circular and linear interactive genome visualization. Bioinformatics 25, 119-120 (2009). https://doi.org/10.1093/bioinformatics/btn578
56 Li, D., Liu, C. M., Luo, R., Sadakane, K. & Lam, T. W. MEGAHIT: an ultra-fast single-node solution for large and complex metagenomics assembly via succinct de Bruijn graph. Bioinformatics 31, 1674-1676 (2015). https://doi.org/10.1093/bioinformatics/btv033
57 Clark, K., Karsch-Mizrachi, I., Lipman, D. J., Ostell, J. & Sayers, E. W. GenBank. Nucleic Acids Res 44, D67-72 (2016). https://doi.org/10.1093/nar/gkv1276
58 Wood, D. E., Lu, J. & Langmead, B. Improved metagenomic analysis with Kraken 2. Genome Biol 20, 257 (2019). https://doi.org/10.1186/s13059-019-1891-0
59 McInnes, L., Healy, J., Saul, N., & GroBberger, L. UMAP: Uniform Manifold Approximation and Projection. Journal of Open Source Software 3 (2018). https://doi.org/10.21105/joss.00861
60 McInnes, L., Healy, J., & Astels, S. hdbscan: Hierarchical density based clustering. Journal of Open Source Software 2, 205 (2017). https://doi.org/10.21105
61 Tatusova, T. et al. NCBI prokaryotic genome annotation pipeline. Nucleic Acids Res 44, 6614-6624 (2016). https://doi.org/10.1093/nar/gkw569
62 Buchfink, B., Xie, C. & Huson, D. H. Fast and sensitive protein alignment using DIAMOND. Nat Methods 12, 59-60 (2015). https://doi.org/10.1038/nmeth.3176
63 Shannon, P. et al. Cytoscape: a software environment for integrated models of biomolecular interaction networks. Genome Res 13, 2498-2504 (2003). https://doi.org/10.1101/gr.1239303

What is claimed is:

1. A therapeutic composition comprising Bacteroides ovatus in a unit dosage of between 1×10⁵ to 9×10⁸ colony forming units (CFU) of the Bacteroides ovatus.

2. The therapeutic composition of claim 1, further comprising a polysaccharide.

3. The therapeutic composition of claim 2, wherein the polysaccharide comprises a dietary-derived polysaccharide.

4. The therapeutic composition of claim 2 or 3, wherein the polysaccharide comprises a xylose-comprising polysaccharide.

5. The therapeutic composition of claim 4, wherein the xylose-comprising polysaccharide comprises arabinoxylan.

6. The therapeutic composition of claim 5, wherein the arabinoxylan comprises wheat arabinoxylan.

7. The therapeutic composition of claim 4, wherein the xylose-comprising polysaccharide comprises xyloglucan.

8. The therapeutic composition of claim 7, wherein the xyloglucan comprises tamarind xyloglucan.

9. A method of treating an intestinal disease in a patient, the method comprising the step of administering to the patient a therapeutically effective amount of the therapeutic composition of any one of claims 1-8.

10. The method of claim 9, wherein the composition comprises a polysaccharide and the polysaccharide is administered concurrently with the Bacteroides ovatus.

11. The method of claim 9, wherein the composition comprises a polysaccharide and the polysaccharide is administered sequentially with the Bacteroides ovatus.

12. The method of any one of claims 9-11, wherein the polysaccharide comprises a dietary-derived polysaccharide.

13. The method of any one of claims 9-12, wherein the polysaccharide comprises a xylose-comprising polysaccharide.

14. The method of claim 13, wherein the xylose-comprising polysaccharide comprises arabinoxylan.

15. The method of claim 14, wherein the arabinoxylan comprises wheat arabinoxylan.

16. The method of claim 13, wherein the xylose-comprising polysaccharide comprises xyloglucan.

17. The method of claim 16, wherein the xyloglucan comprises tamarind xyloglucan.

18. The method of any one of claims 9-17, wherein the intestinal disease comprises acute gastrointestinal graft versus host disease, an antibiotic-mediated microbiome injury, or intestinal inflammation.

19. The method of claim 18, wherein when the disease is an antibiotic-mediated microbiome injury, the patient has received or will receive an antibiotic.

20. The method of claim 18, wherein the antibiotic comprises a broad spectrum antibiotic.

21. The method of claim 19 or 20, wherein the antibiotic comprises cefepime, daptomycin, linezolide, penicillin, metronidazole, sulfamethoxazole, trimethoprim, vancomycin, and/or clindamycin.

22. The method of claim 19 or 20, wherein the antibiotic comprises a carbapenem, a macrolide, a quinolone, and/or an aminoglycoside.

23. The method of claim 22, wherein the carbapenem comprises meropenem.

24. A method of preventing complications from hematopoietic stem cell transplantation in a patient, the method comprising the step of administering to the patient a therapeutically effective amount of the therapeutic composition of any one of claims 1-8.

25. The method of claim 24, wherein the composition comprises a polysaccharide and the polysaccharide is administered concurrently with the Bacteroides ovatus.

26. The method of claim 24, wherein the composition comprises a polysaccharide and the polysaccharide is administered sequentially with the Bacteroides ovatus.

27. The method of claim 26, wherein the polysaccharide comprises a dietary-derived polysaccharide.

28. The method of claim 26 or 27, wherein the polysaccharide comprises a xylose-comprising polysaccharide.

29. The method of claim 28, wherein the xylose-comprising polysaccharide comprises arabinoxylan.

30. The method of claim 29, wherein the arabinoxylan comprises wheat arabinoxylan.

31. The method of claim 28, wherein the xylose-comprising polysaccharide comprises xyloglucan.

32. The method of claim 31, wherein the xyloglucan comprises tamarind xyloglucan.

33. The method of any one of claims 24-32, wherein the hematopoietic stem cell transplantation comprises allogeneic hematopoietic stem cell transplantation.

34. A method for inducing mucus degradation in intestines of a patient, the method comprising the step of administering to the patient a therapeutically effective amount of the therapeutic composition of any one of claims 1-8.

35. The method of claim 34, wherein the composition comprises a polysaccharide and the polysaccharide is administered concurrently with the Bacteroides ovatus.

36. The method of claim 34, wherein the composition comprises a polysaccharide and the polysaccharide is administered sequentially with the Bacteroides ovatus.

37. The method of claim 36, wherein the polysaccharide comprises a dietary-derived polysaccharide.

38. The method of claim 36 or 37, wherein the polysaccharide comprises a xylose-comprising polysaccharide.

39. The method of claim 38, wherein the xylose-comprising polysaccharide comprises arabinoxylan.

40. The method of claim 39, wherein the arabinoxylan comprises wheat arabinoxylan.

41. The method of claim 38, wherein the xylose-comprising polysaccharide comprises xyloglucan.

42. The method of claim 41, wherein the xyloglucan comprises tamarind xyloglucan.

43. A method of reducing Bacteroides thetaiotaomicron (BT) in a patient, the method comprising the step of administering to the patient a therapeutically effective amount of the therapeutic composition of any one of claims 1-8.

44. The method of claim 43, wherein the composition comprises a polysaccharide and the polysaccharide is administered concurrently with the Bacteroides ovatus.

45. The method of claim 43, wherein the composition comprises a polysaccharide and the polysaccharide is administered sequentially with the Bacteroides ovatus.

46. The method of claim 45, wherein the polysaccharide comprises a dietary-derived polysaccharide.

47. The method of claim 45 or 46, wherein the polysaccharide comprises a xylose-comprising polysaccharide.

48. The method of claim 47, wherein the xylose-comprising polysaccharide comprises arabinoxylan.

49. The method of claim 48, wherein the arabinoxylan comprises wheat arabinoxylan.

50. The method of claim 47, wherein the xylose-comprising polysaccharide comprises xyloglucan.

51. The method of claim 50, wherein the xyloglucan comprises tamarind xyloglucan.

52. A method comprising administering to a patient the therapeutic composition of any one of claim 1-8.

53. The method of claim 52, wherein the patient has, or is at risk of developing, graft versus host disease (GVHD)

54. The method of claim 53, wherein the patient has, or is at risk of developing, acute gastrointestinal GVHD.

55. The method of any one of claims 52-54, wherein the patient is steroid refractory.

56. The method of claim 52, wherein the patient has, or is at risk of developing, an intestinal disease.

57. The method of claim 56, wherein the intestinal disease is an antibiotic-mediated microbiome injury or intestinal inflammation.

58. The method of claim 57, wherein when the disease is an antibiotic-mediated microbiome injury, the patient has received or will receive an antibiotic.

59. The method of claim 58, wherein the antibiotic comprises a broad spectrum antibiotic.

60. The method of claim 58 or 59, wherein the antibiotic comprises cefepime, daptomycin, linezolide, penicillin, metronidazole, sulfamethoxazole, trimethoprim, vancomycin, and/or clindamycin.

61. The method of any one of claims 58-60, wherein the antibiotic comprises a carbapenem, a macrolide, a quinolone, and/or an aminoglycoside.

62. The method of claim 61, wherein the carbapenem comprises meropenem.

63. The method of claim 52, wherein the patient has received or will receive an antibiotic.

64. The method of claim 63, wherein the antibiotic comprises a broad spectrum antibiotic.

65. The method of claim 62 or 64, wherein the antibiotic comprises cefepime, daptomycin, linezolide, penicillin, metronidazole, sulfamethoxazole, trimethoprim, vancomycin, and/or clindamycin.

66. The method of claim 62 or 64, wherein the antibiotic comprises a carbapenem, a macrolide, a quinolone, and/or an aminoglycoside.

67. The method of claim 66, wherein the carbapenem comprises meropenem.

68. The method of claim 52, wherein the patient has received or will receive a stem cell treatment.

69. The method of claim 68, wherein the stem cell treatment comprises a hematopoietic stem cell transplantation.

70. The method of claim 69, wherein the hematopoietic stem cell transplantation comprises allogeneic hematopoietic stem cell transplantation.

71. The method of any one of claims 52-70, wherein the patient has an altered microbiome profile compared to a standard.

72. The method of claim 71, wherein the altered microbiome profile comprises a less diverse microbiome profile than a standard.

73. The method of claim 71 or 72, wherein the altered microbiome profile comprises a higher abundance of Enterococcus, Citrobacter, Streptococcus, Staphylococcus, and/or Enterobacter bacteria compared to a standard.

74. The method of any one of claims 71-73, wherein the altered microbiome profile comprises a lower abundance of Bacteroides, Prevotella, Faecalibacterium, and/or UBA1819 bacteria compared to a standard.

75. The method of any one of claims 71-74, wherein the standard comprises a microbiome profile of a healthy individual.

76. The method of any one of claims 71-75, wherein the standard comprises a microbiome profile of a patient found responsive to a steroid.

77. A method for prognosing responsiveness to a steroid therapy in a patient, the method comprising the steps of;
obtaining a microbiome profile from the patient; and
comparing the microbiome profile to a standard.
wherein the patient has, or will receive, a stem cell therapy.

78. The method of claim 77, wherein the standard comprises a microbiome profile of a healthy individual.

79. The method of claim 77 or 78, wherein the standard comprises a microbiome profile of a patient found responsive to a steroid.

80. The method of any one of claims 77-79, wherein the patient is prognosed to be steroid-refractory when the microbiome profile from the patient comprises a higher abundance of Enterococcus, Citrobacter, Streptococcus, Staphylococcus, and/or Enterobacter bacteria compared to the standard.

81. The method of any one of claims 77-80, wherein the patient is prognosed to be steroid-refractory when the microbiome profile from the patient comprises a lower abundance of Bacteroides, Prevotella, Faecalibacterium, and/or UBA1819 bacteria compared to the standard.

82. The method of any one of claims 77-81, wherein the patient is prognosed to be steroid-refractory when the microbiome profile is less diverse compared to the standard.


SEQUENCE LISTING (4 sequences)

SEQ ID NO: 1 - DNA, 253 bp, Bacteroides ovatus
  source 1..253: mol_type = genomic DNA
tacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgttactgacactgatgctcgaaagtgtgggtatcaaacang

SEQ ID NO: 2 - DNA, 253 bp, Bacteroides ovatus
  source 1..253: mol_type = genomic DNA
tacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacang

SEQ ID NO: 3 - DNA, 253 bp, Bacteroides ovatus
  source 1..253: mol_type = genomic DNA
tacggaggatccgagcgttatccggatttattgggtttaaagggagcgtaggtggattgttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagctcactagactgttactgacactgatgctcgaaagtgtgggtatcaaacang

SEQ ID NO: 4 - DNA, 253 bp, Bacteroides ovatus
  source 1..253: mol_type = genomic DNA
tacgtagggggcaagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgttactgacactgatgctcgaaagtgtgggtatcaaacagg